(19) United States
(12) Patent Application Publication
(10) Pub. No.: US 2025/0304525 A1
(43) Pub. Date: Oct. 2, 2025

(54) METHOD FOR MANUFACTURE OF ISOPHORONEDIAMINE

(71) Applicant: BASF SE, Ludwigshafen am Rhein (DE)

(72) Inventors: Alfred KRAUSE, Ludwigshafen am Rhein (DE); Marina-Eleni STAVROU, Ludwigshafen am Rhein (DE); Martin SCHLODERER, Ludwigshafen am Rhein (DE)

(21) Appl. No.: 18/277,233

(22) PCT Filed: Feb. 10, 2022
(86) PCT No.: PCT/EP2022/053216

§ 371 Date: Aug. 15, 2023

(30) Foreign Application Priority Data
Feb. 16, 2021 (EP) 21157300.1

(51) Int. Cl. C07C 209/86; C07C 209/48; C07C 209/84; C07C 263/10; C08G 18/75

(52) U.S. Cl. CPC C07C 209/86; C07C 209/48; C07C 209/84; C07C 263/10; C08G 18/755; C07C 2601/14

(57) ABSTRACT

A process for the manufacture of isophorone diamine (IPDA), comprising the steps of:
a) providing a feed stream comprising trans-IPDA, cis-IPDA, isophorone nitrile amine (IPNA), components having a lower boiling point than trans-IPDA and components having a higher boiling point than IPNA, including isophorone amino alcohol (IPAA) and components having a higher boiling point than IPAA;
b) separating the feed stream into
   (i) a fraction (ii) comprising a higher mass fraction of cis-IPDA content, compared to the feed stream;
   (ii) a fraction (iii) comprising a higher mass fraction of IPNA compared to the feed stream and a higher mass fraction of components having a boiling point higher than the boiling point of IPNA, including IPAA and components having a higher boiling point than IPAA, compared to the feed stream and optionally IPDA;
c) further separating fraction (iii) into
   (iii) a fraction (iii-1) comprising a higher mass fraction of IPDA, compared to fraction (iii); and/or
   (iv) a fraction (iii-3) comprising a higher mass fraction of IPAA, compared to fraction (iii).

The present invention relates to a process for the manufacture of isophorone diamine (IPDA).

IPDA is used as a starting product for preparing isophorone diisocyanate (IPDI), an isocyanate component for polyurethane systems, as an amine component for polyamides and as a hardener for epoxy resins.

IPDA is usually prepared in a multistage process starting from isophorone (IP). In a first step, hydrogen cyanide (HCN) is added to IP to obtain the corresponding isophorone nitrile (IPN). In a further step, IPN is converted to IPDA by converting the carbonyl group of IPN to an amino group and the nitrile group to an aminomethyl group in the presence of ammonia, hydrogen and hydrogenation catalysts. The second step can be divided into further steps, in which the carbonyl group of IPN is first converted with ammonia (NH3) to the corresponding isophorone nitrile imine (IPNI) in the presence of an imination catalyst. In a subsequent step, IPNI is then hydrogenated in the presence of a hydrogenation catalyst to obtain IPDA.

In the preparation of IPDA, it is very important not only to achieve a high product yield of IPDA but also to control the isomer ratio between cis-IPDA and trans-IPDA, since these isomers have different reactivities. According to DE-A-4211454, IPDA with a high cis-trans-ratio (CTR) of 75:25 and more are preferred in applications, which require a short pot-life and a short curing temperature. This is the case in most epoxy and PUR-applications. IPDA with a high CTR is therefore commercially preferred. Some customers specify a CTR of >75:25 for their applications.

The CTR in IPDA is influenced by many factors.

One prior art process discloses that a high CTR can be achieved by a two-stage conversion of IPNI by controlling the temperature in the respective stages (EP 0394968).

According to DE 19507398 and DE19747913, the addition of a base or a basic compound to the hydrogenative amination also has an influence on the isomer ratio.

WO2008077852 further teaches that the time of addition of the base to the hydrogenation step can also lead to an increase of the CTR.

High CTRs are also achieved when the hydrogenation reaction is carried out with basic catalysts (DE4010227 and EP0623585).

An increase of the CTR was also reported, when the reductive amination was carried out in the presence of an acid (DE19756400).

Even if the reaction conditions are carefully selected to control the CTR, e.g. by the choice of catalyst, it is possible that the CTR decreases when the catalysts employed in the reaction ages and loses at least some of its selectivity towards cis-IPDA.

To counterbalance the decrease of the CTR, it is sometimes proposed in the state of the art to subject the produced IPDA to an isomerization step (WO2016143538, EP1529028).

DE10236674 teaches a method for enhancing the CTR by distillation. The method makes uses of the principle that the cis-isomer of IPDA has a higher boiling point than the trans-isomer. A crude IPDA having a CTR of less than 73:27 is separated into a fraction having a CTR of <66:34, which may be drawn-off at the top of the distillation column, and a fraction having a CTR of >73:27, which is typically drawn-off as a side-offtake. The distillation parameters, such a reflux and temperature, are controlled to achieve the quality of the desired fractions. The process according to DE10236674 has the advantage, that an IPDA fraction having a high CTR can be obtained, which can be used in applications requiring a high CTR, while further obtaining a fraction with a lower CTR, which can be used in application in which the CTR is of lower importance. Using the process of DE10236674, nearly the entire yield of IPDA produced can be utilized without substantial losses.

However, it was surprisingly found that the process according to DE10236674 has its limits, when additional isophorone nitrile amine (IPNA) is present in admixture with IPDA.

IPNA is an intermediate product formed during the hydrogenation of IPNI if only the imine group is hydrogenated, but not the nitrile group. IPNA has a similar boiling point compared to IPDA and is therefore difficult to separate from IPDA.

It was found, that IPDA fractions enriched in cis-isomer and having a low IPNA content show an improved performance in down-stream applications of IPDA. Especially good properties are obtainable, when the maximum content of IPNA in the IPDA-sales product is less than 0.2% by weight.

When applying the process of DE10236674, it was found that such low IPNA-specification can only be reached by operating the column in which the IPDA is enriched with a high reflux ratio. An increase of the reflux ratio enhances the undesired side effect of increasing the concentration of cis- and trans-IPDA in the sump of the separation column resulting in undesirable losses of IPDA.

The object of the present invention was to provide a process for the manufacture of IPDA yielding an IPDA fraction having a high CTR and a low content of IPNA while minimizing the loss of IPDA. A further object of the invention was to increase the overall process yield of IPDA and the IPDA process recovery. Further, it was an object of the invention (i) to decrease the specific energy demand, (ii) to achieve a reduction in the consumption of crude materials and (iii) to reduce the carbon dioxide footprint to create a more sustainable process. A still further object of the invention was to provide a process with the potential of obtaining the additional value product isophorone amino alcohol (IPAA) without substantially increasing the IPDA product loss and without substantially increasing the specific energy demand of the separation process. IPAA is an important intermediate in various fields of use. For example, it serves as a precursor of pharmacological products, especially in the field of influenza prophylaxis (WO2011/095576). Further applications include use in polymers, anti-corrosives and stabilizers (DE1229078).

The object of the present invention was achieved by
a process for the manufacture of isophorone diamine (IPDA), comprising the steps of:
a) providing a feed stream comprising trans-IPDA, cis-IPDA, isophorone nitrile amine (IPNA), components having a lower boiling point than trans-IPDA and components having a higher boiling point than IPNA, including isophorone amino alcohol (IPAA) and components having a higher boiling point than IPAA;
b) separating the feed stream into
(i) a fraction (ii) comprising a higher mass fraction of cis-IPDA content, compared to the feed stream;
(ii) a fraction (iii) comprising a higher mass fraction of IPNA compared to the feed stream and a higher mass fraction of components having a boiling point higher than the boiling point of IPNA, including IPAA and components having a higher boiling point than IPAA, compared to the feed stream and optionally IPDA;
c) further separating fraction (iii) into
(iii) a fraction (iii-1) comprising a higher mass fraction of IPDA, compared to fraction (iii); and/or
(iv) a fraction (iii-3) comprising a higher mass fraction of IPAA, compared to fraction (iii).

Surprisingly, it was found that the use of the IPDA-fractions prepared by the inventive process results in improved properties in down-stream applications, which are probably attributable to the depletion of IPNA in the final sales product. It was found that IPDA-fractions having the required properties can be prepared, if crude IPDA (as described below) is separated into, a fraction (ii) having an increased mass fraction of cis-IPDA. The process of the present invention requires that a further fraction (iii) is separated, which is enriched in IPNA and other components having a boiling point higher than IPNA, including IPAA and components having a higher boiling point than IPAA.

Optionally, if components with a lower boiling point than trans-IPDA are present in the feed, a further fraction (iv) is separated which comprises these lower boiling components.

Fraction (iv) is preferably further separated in an organic phase (iv-a) and an aqueous phase (iv-b).

In a preferred embodiment an additional fraction (i) comprising a higher mass fraction of trans-IPDA, compared to the feed stream, is separated. In this embodiment, it is possible to enrich the content of cis-IPDA in fraction (ii), if the CTR in the feed stream is lower than required for the intended use or application. Many applications require that the CTR in the final IPDA-product is 70:30 or more, preferably 73:27 or more and more preferably 75:25 or more.

Separating the feed stream of crude IPDA into fractions (ii) and (iii) and optionally (iv) and optionally (i) allows to operate the separation process to obtain the fractions (i) and/or (ii) with a sufficiently low IPNA content and a stream (iii) enriched in IPNA and IPAA, which can be further separated to reclaim IPDA which may still be present in stream (iii).

Due to the possibility of recovery of lost IPDA from the higher boiling waste stream (fraction (iii)), the present invention even allows the conversion of IPN to IPDA without a post-hydrogenation reactor, which is often required to decrease the yield of undesired IPNA during the conversion of IPN to IPDA.

The present invention further allows to separate the value product IPAA present in stream (iii). IPAA is an important intermediate in various fields of use. For example, it serves as a precursor of pharmacological products, especially in the field of influenza prophylaxis (WO2011/095576). Further applications include use in polymers, anti-corrosives and stabilizers (DE1229078).

In a one embodiment of the invention, the fraction (iii) is further separated in into one or more of the following fractions:
a fraction (iii-2) comprising a higher mass fraction of IPNA compared to fraction (iii); and/or
a fraction (iii-4) comprising a higher mass fraction of components having a boiling point higher than IPAA.

In one preferred embodiment, fraction (iii) is separated into fractions (iii-1); and

a fraction (iii-b), which comprises fractions (iii-2), (iii-3) and (iii-4).

This embodiment allows an improved IPDA recovery.

Preferably, fraction (iii-b) is further separated in fractions (iii-2), (iii-3) and (iii-4), which allows for an additional IPAA recovery.

In a second preferred embodiment, fraction (iii) is separated into fractions (iii-1), (iii-2), (iii-3) and (iii-4) in a single column, allowing to reduce the number of columns and to reduce investment costs.

The two aforementioned preferred embodiments are especially useful if the CTR in the raw IPDA is 80:20 or less, preferably 75:25 or less, more preferably 73:27 or less and most preferably 70:30 or less, allowing not only IPDA and/or IPAA recovery but also the production of IPDA fractions (i) and/or (ii) with a low IPNA content.

In a third preferred embodiment, fraction (iii) is separated in a fraction (iii-a), comprising fraction (iii-1) and fraction (iii-2); and

into fraction (iii-3); and

into fraction (iii-4).

This embodiment allows the recovery of IPAA and the production of the IPDA fraction (ii) with a low IPNA content. This embodiment is particularly useful if the CTR of the raw IPDA is already 70:30 or higher, preferably 73:27 of higher, more preferably 75:25 or higher and even more preferably 80:20 or higher. If the CTR in the raw IPDA is in the aforementioned range, it is usually not necessary to separate off an additional fraction (i), because the CTR in fraction (ii) is already in the commercially required range.

The feed stream comprising IPDA entering a process of the invention may be obtained by either (A) converting IPN in the presence of NH3, H2 and a hydrogenation catalyst in a single step or (B) converting IPN in the presence of NH3, H2 and a hydrogenation catalyst in at least two stages, by first converting IPN fully or partly with NH3 in the presence of an imination catalyst to obtain isophoronenitrileimine (IPNI) and further reacting IPNI with hydrogen in the presence of a hydrogenation catalyst and optionally ammonia.

Methods for preparing IPDA are known in the art.

Preferably IPDA is prepared in two stage process by a) converting IPN with ammonia to IPNI and b) reacting the product from step a) with hydrogen in the presence of a hydrogenation catalyst and ammonia.

### Imination

The first stage (imination) of the two-staged process of converting IPN to IPDA is usually conducted at temperature from 20 to 150° C., preferably 30 to 100° C. and more preferably 50 to 90° C. and a pressure of 50 to 300 bar, preferably 100 to 250 bar and more preferably 150 to 220 bar. Suitable imination catalysts are usually acidic oxides, preferably alumina, titania, zirconia and silica. The catalyst loading is preferably in the range of 0.01 to 10, more preferably 0.05 to 7 and even more preferably 0.1 to 5 kg IPN per kg catalyst.

The molar ratio of NH3 to IPN is usually in the range of 5:1 to 500:1, preferably 10:1 to 400:1 and more preferably 20:1 to 300:1.

The imination can be optionally conducted in the presence of a solvent, such as alcohols or ethers, in particularly THF, ethanol or butanol. Most preferably, the imination is not conducted in the presence of a solvent.

The imination can be conducted in one or more pressurized reaction vessels, most preferably the one or more pressurized reaction vessels are one or more tubular reactors where the imination catalyst is arranged in a fixed bed. Preferably the imination is conducted in 1 to 3, more preferably 1 to 2 and even more preferably in one reactor.

The reaction conditions, such as temperature, catalyst, pressure, reactor geometry, are selected in such a manner that the conversion of IPN to IPNI is preferably 80% or more, more preferably 90% or more and most preferably 95% or more.

### Hydrogenation

The effluent from the imination step is preferably converted in a second step with hydrogen in the presence of a hydrogenation catalyst and ammonia.

Preferably, the amount of ammonia present during the previous imination step is selected in such a manner, that the ammonia concentration during the hydrogenation step is in a suitable range. A suitable molar ratio of ammonia to IPNI in the hydrogenation step is about 5:1 to 500:1, preferably 10:1 to 400:1 and most preferably 20:1 to 300:1. Additional ammonia can also be optionally added to bring the ammonia concentration into the aforementioned ranges.

The hydrogenation step is conducted in the presence of hydrogen.

The molar ratio between hydrogen and IPNI is preferably in the range of 3:1 to 10000:1, more preferably 4:1 to 5000:1 and most preferably 5:1 to 1000:1.

In a preferred embodiment, hydrogen is added after the imination step. It is however possible, that hydrogen is added prior to the imination step because the imination is usually carried out in the presence of catalysts which do not catalyse the hydrogenation of the imine or nitrile group. The hydrogenation can also be conducted in one or more pressurized reaction vessels.

Most preferably the one or more pressurized reaction vessels are one or more tubular reactors where the hydrogenation catalyst is arranged in a fixed bed. Preferably the hydrogenation is conducted in 1 to 3, more preferably 1 to 2 and even more preferably in one single reactor, preferably a fixed bed reactor.

The temperature during the hydrogenation is usually in the range of 40 to 200° C., preferably 50 to 150° C., more preferably 60 to 140° C. and most preferably 60 to 130° C. and a pressure of 50 to 300 bar, preferably 100 to 250 bar and more preferably 150 to 220 bar.

The catalyst load during the hydrogenation is also in the range of 0.01 to 10, preferably 0.05 zo 7, more preferably 0.1 to 5 kg IPNI per kg catalyst per hour.

The hydrogenation is preferably carried out in the presence of hydrogenation catalysts, which usually comprise metals or semimetals from groups 1 to 17 of the Periodic Table as well as the rare earth metals.

Preferred catalyst elements are Ni, Co, Fe, Cu, Ru. Hydrogenation catalysts may also comprise Cr, Cu, Mo. Wo and/or Re.

Preferred hydrogenation catalysts comprise one or more of Ru and Co,

The hydrogenation catalysts can of the so-called Raney-type or the metal-oxide type. Preferred Raney-type catalysts are Raney-Co-catalysts. The Raney-type catalysts may be supported or unsupported. Suitable Raney-Catalysts are further described in EP1207149, EP 2649042WO2008107226, WO2014086039 and WO2016120235,

The hydrogenation catalysts can also be of the metal-oxide type.

Metal-oxide catalysts are preferably obtained by precipitation of soluble salts of the catalyst elements in the presence of catalyst supports to obtain the corresponding hydroxides, carbonates and oxides and which are usually transformed to the corresponding oxides during a calcination step. The precipitation step may also be conducted without the presence of support materials. Alternatively, the hydrogenation catalyst may be produced by impregnation of a catalyst support with soluble salts of the metals.

The metal-oxides catalysts are usually reduced in the presence of hydrogen prior to their use in the hydrogenation step. The reduced catalysts may be passivated by subjecting the reduced catalysts to an oxygen comprising gas in order to form a passivating and protective oxide layer which allows for safe handling and storage. The passivated catalysts may be reduced or activated prior to their use in the hydrogenation step. Activation and reduction of the metal oxide catalyst is preferably performed in the same reactor, in which the hydrogenation IPNI is performed. The reduction or passivation step may occur prior to the hydrogenation step, but it is also possible to reduce or activate the metal oxide catalysts in-situ during the hydrogenation of IPNI. The unreduced or inactivated catalyst is then transformed into its reduced form by the hydrogen present during the hydrogenation reaction.

Preferred supports are alumina, including but not limited to transitional alumina and non-traditional alumina, titania, zirconia, silica, magnesia, calcium oxide and mixtures thereof.

In a further preferred embodiment, the basicity of the effluent from the imination stage is increased prior or during the subsequent hydrogenation step.

An increase of the basicity can be achieved by the addition of basic compounds or using hydrogenation catalysts which are supported on a basic support. Preferably the basic support comprises elements, such as oxides, of the alkaline metals, preferably Li, Na and K, the alkaline earth metals, preferably Mg and Ca or comprises basic minerals, preferably hydrotalcite, chrysotile or sepiolite.

Preferred basic catalysts are those which are disclosed in WO 2008077852.

In a most preferred embodiment, unsupported hydrogenation catalysts comprising 55 to 98 weight percent of Co, 0.2 to 15 weight percent of P, 0.2 to 15 weight percent of Mn and 0.2 to 15 weight percent of alkali, in particularly Na, are used. Details regarding the specification and production of such catalysts can be found in DE4325847.

Basic compounds can also be added in form of their solutions.

Suitable basic compounds are usually compounds of basic metals, in particularly the oxides, hydroxides or carbonates of alkaline metals, alkaline earth metals or the rare earth metals.

Other suitable basic compounds are ammonium hydroxide and amines.

Preferred basic compounds are oxides, hydroxides and carbonate, in particular Li₂O, Na₂O, K₂O, Rb₂O, Cs₂O, LiOH, NaOH, KOH, RbOH, CsOH, Li₂CO3, Na2CO₃, K₂CO₃, Cs₂CO₃, Rb₂CO₃, MgO, CaO, SrO, BaO, Mg(OH)₂, Ca(OH)₂, Sr(OH)₂, Ba(OH)₂, MgCO₃, CaCO₃, SrCO₃ or

BaCO₃. In particularly preferred basic compounds are LiOH, NaOH and KOH.

The basic compounds are preferably added in form of their solutions in water or other suitable solvents, such as alkanols, like C₁-C₄-alkanols, in particularly methanol or ethanol, or ethers, such as cyclic ether, in particularly THF or dioxane. Preferably the basic compounds are added in form of their aqueous solutions.

The concentration of basic compounds in water or other suitable solvents is usually around 0.01 bis 20 percent by weight, preferably 0, 1 bis 10 percent by weight and more preferably 0.2 bis 5 percent by weight.

The amount of added basic compound is usually determined in such a way as to yield a molar ratio of basic compound to IPNI is in the range of 100:1 000 000 to 10 000:1 000 000 and more preferably 200:1 000 000 to 1000:1 000 000.

Further details regarding the addition of basic compounds prior to the hydrogenation step is disclosed in EP729937 or EP913387, whereas further details regarding the addition of basic compounds during the hydrogenation stage is disclosed in WO 2008077852.

The effluent from the conversion of IPN to IPDA, which is either conducted in a single step or as a two-stage process, comprising imination and hydrogenation as set out above, usually comprises
cis-IPDA,
trans-IPDA,
IPNA,
hydrogen,
ammonia,
components having a boiling point higher than IPNA, including IPAA and components having a boiling point higher than IPAA, and
optionally components having a boiling point lower than trans-IPDA.

When the effluent comprises hydrogen and ammonia, the effluent from the reductive amination is usually worked-up by first separating hydrogen and ammonia.

The removal of hydrogen is preferably carried out by subjecting the effluent to a high pressure separator which usually results in the separation of a gaseous phase, comprising hydrogen and some ammonia, and a liquid phase comprising ammonia, cis-IPDA, trans-IPDA, IPNA, components having a boiling point higher than IPNA and optionally, components having a boiling point lower than trans-IPDA.

The high-pressure separator is usually operated at pressure slightly lower than the pressure at which the hydrogenation reactor is operated, preferably of 2 to 350 bar, preferably 10 to 240 bar and more preferably 30 to 210 bar. The gaseous phase is preferably compressed to the reaction pressure and recycled to the hydrogenation reactor. The liquid phase from the high-pressure separator is usually subjected to one or more separation step, in which ammonia is separated from the rest of the components, Such separation steps may comprise one or more flash operations, stripping operations or distillation operations to obtain an ammonia fraction and the crude IPDA fraction. In a preferred embodiment, ammonia is separated in one or more distillation columns.

The distillation column is usually operated at pressures in the range of 5 to 50, preferably 10 to 40 and more preferably 15 to 30 bar. In a more preferred embodiment, a second ammonia removal step is conducted after the first ammonia removal step. Such a second step is preferably conducted in another distillation column usually operated at 1.5 to 20, preferably 2 to 15 and more preferably 3.5 to 10 bar.

The composition of the effluent from the reductive amination of IPN after removal of ammonia and/or hydrogen is usually denoted as “crude IPDA”.

The crude IPDA usually comprises:
72.9 to 95 percent by weight: IPDA (cis and trans)
5-13 percent by weight: water
0-4 percent by weight: components having a boiling point lower than trans-IPDA
0 to 0.1 percent by weight: IPNA
0 to 4 percent by weight: IPAA
0 to 6 percent by weight: components having a boiling point higher than IPAA.

Preferably the crude IPDA comprises:
78.9 to 93.3 percent by weight: IPDA (cis and trans)
6 to 11 percent by weight: water
0.5 to 3 percent by weight: components having a boiling point lower than trans-IPDA
0 to 0.1 percent by weight: IPNA
0.5 to 3 percent by weight: IPAA
0.2 to 4 percent by weight: components having a boiling point higher than IPAA

More preferably, the crude IPDA comprises
83.6 to 91.3 percent by weight: IPDA (cis and trans)
7-10 percent by weight: water
0.5-2.5 percent by weight: components having a boiling point lower than trans-IPDA
0.1 to 0.4 percent by weight: IPNA
1 to 3 percent by weight: IPAA
0.1 to 0.5 percent by weight: components having a boiling point higher than IPAA

According to the invention a feed stream of crude IPDA is subjected to further separation steps to obtain
(i) a fraction (ii) comprising a higher cis-IPDA content, compared to the feed stream;
(ii) a fraction (iii) comprising IPDA and a fraction comprising a higher content of components having a boiling point equal or higher than the boiling point of IPNA, compared to the feed stream, and

further separating fraction (iii) into
(iii) a fraction (iii-1) comprising a higher content of IPDA, compared to fraction (iii); and/or
(iv) a fraction (iii-3) comprising a higher content of IPAA, compared to fraction (iii).

Preferably, fraction (iii) is further separated into one or more of the following fractions: a fraction ((iii-2) comprising a higher mass fraction of IPNA compared to fraction (iii); and/or a fraction ((iii-4) comprising a higher mass fraction of components having a boiling point higher than IPAA compared to fraction (iii).

If crude IPDA comprises components having a boiling point lower than trans-IPDA, then preferably a further fraction (iv), comprising these low boiling components, is separated-off

In a preferred embodiment, a fraction i) comprising a higher mass fraction of trans-IPDA content, compared to the feed stream, is separated. This embodiments is especially useful if the CTR in the raw IPDA is 80:20 or less, preferably 75:25 or less, more preferably 73:27 or less and most preferably 70:30 or less, because a fraction (ii) enriched in cis-IPDA, compared to the raw IPDA, can be obtained.

In a further preferred embodiment, fraction (iii) is separated into a fraction (iii-1) and a fraction (iii-b), comprising fractions (iii-2), (iii-3) and (iii-4).

In a further preferred embodiment, fraction (iii-b) is separated into fractions (iii-2), (iii-3) and (iii-4) in an additional column.

In still another preferred embodiment, fraction (iii) is separated into fractions (iii-1), (iii-2), (iii-3) and (iii-4) in a single column.

In a further preferred embodiment fraction (iii) is separated into a fraction (iii-a), comprising fractions (iii-1) and (iii-2); and a fraction (iii-3) and a fraction (iii-4) in a single column.

The separation into the desired fractions can be achieved by different interconnections of two or more distillation columns.

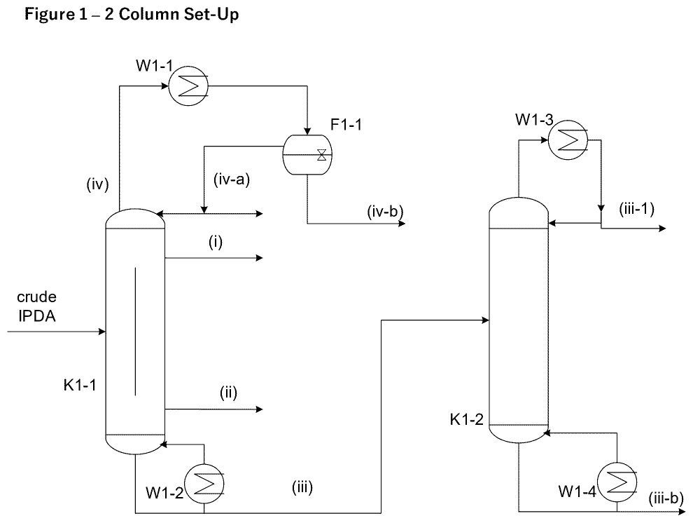

Preferred embodiments of interconnected distillation columns are demonstrated in FIGS. 1 to 8.

Embodiment According to FIG. 1:

In a preferred embodiment, the separation of crude IPDA can be carried out in a set-up comprising two distillation columns. In this embodiment the first column is a dividing wall column K1-1 and the second column K1-2 (IPDA recovery column) is a distillation column. In the diving wall column K1-1, crude IPDA is preferably introduced as a middle stream and fraction (iv) is preferably drawn-off at the top of K1-1, fraction (i) is preferably drawn-off as a top side stream, fraction (ii) is preferably drawn-off as a bottom side stream and fraction (iii) is preferably drawn-off from the sump of K1-1. Fraction (iii) is then fed into column K1-2 in which fraction (iii-1) is preferably drawn-off at the top and fraction (iii-b) is preferably drawn-off from the sump of K1-2.

K1-1 is preferably operated at:
a pressure in the range of 10 to 1000 mbar, preferably 50 to 700 mbar, more preferably 50 to 150 mbar;
a top temperature of preferably 10 to 120° C., more preferably 15 to 100° C., more even more preferably 20 to 70° C.;
a bottom temperature of preferably 150 to 300° C., more preferably 170 to 250° C., and even more preferably 150 to 195° C.

The feed of crude IPDA is preferably introduced into the middle of the column, preferably at a position at around 30 to 70% of the number of theoretical trays, more preferably 40 to 60% and even more preferably 45 to 55%.

Fraction (iv) is preferably withdrawn at the top of the column, where it is preferably condensed in a condenser W1-1, which is preferably operated at around 5 to 110, more preferably 10 to 90, and even more preferably 15 to 60° C. The condensed phase is preferably fed to a phase separator F1-1 and preferably separated into a lighter organic phase (iv-a) and a heavier aqueous phase (iv-b). The lighter organic phase (iv-a) is preferably partially discarded and partially fed back to the column as reflux.

The organic reflux ratio (reflux to organic distillate) is preferably in the range of 10 to 300, more preferably 20 to 280 and more preferably 100 to 250.

Fraction (i) is preferably withdrawn as a side-take-off, in the other side of the dividing wall as the feed-side and above the feed height.

Fraction (ii) is preferably withdrawn as a side-take-off, in the other side of the dividing wall as the feed-side and bellow the feed height.

The bottom of the column K1-1 is preferably connected to a reboiler W1-2 and fraction (iii) is preferably withdrawn as a bottom product from the bottom of the column.

The reboiler W1-2 is preferably a kettle type reboiler, a thermosyphon reboiler, a fired reboiler or a forced circulation reboiler, most preferably a kettle type reboiler or a thermosyphon reboiler.

The column K1-2 is preferably operated at:
a pressure in the range of 5 to 300 mbar, preferably 7 to 100 mbar, more preferably 10 to 60 mbar;
a top temperature of preferably 80 to 210° C., more preferably 110 to 180° C., more even more preferably 120 to 170° C.;
a bottom temperature of preferably 130 to 270° C., more preferably 140 to 230° C., and even more preferably 150 to 210° C.

The feed of fraction (iii) from K1-1 is preferably introduced into the middle of the column K1-2, preferably at a position at around 5 to 70% of the number of theoretical stages, more preferably 5 to 50% and even more preferably 10 to 40% from the sump of the column.

The bottom of the column is preferably connected to a reboiler W1-4 and fraction (iii-b) is preferably withdrawn as a bottom product from K1-2.

The reboiler W1-4 is preferably a kettle type reboiler, a thermosyphon reboiler, a fired reboiler or a forced circulation reboiler, most preferably a kettle type reboiler or a thermosyphon reboiler. Fraction (iii-1) is preferably drawn-off as a top product from column K1-2 and condensed at condenser W1-3. The condenser W1-3 is usually operated at around 40 to 190, preferably 80 to 180, more preferably 100 to 160° C. Preferably, a part of fraction (iii-1) is refluxed back to the column K1-2.

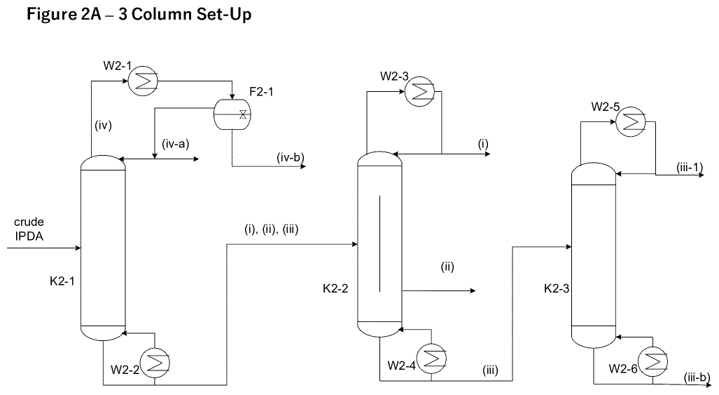

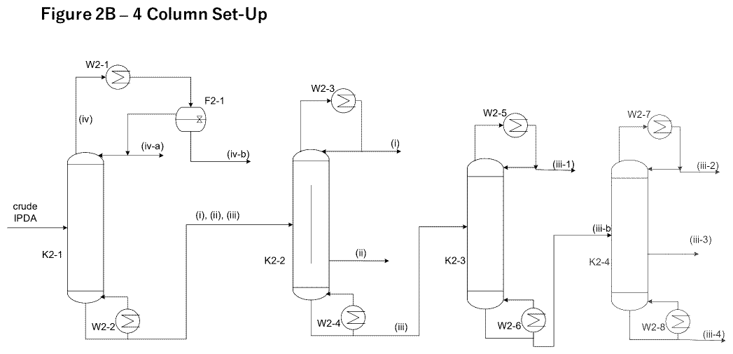

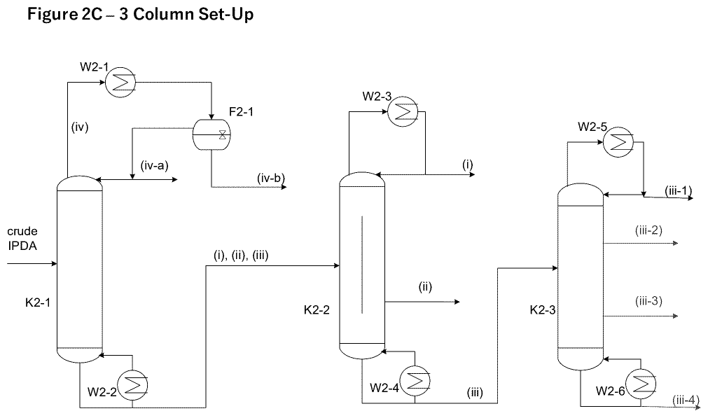

Embodiment according to FIG. 2A:

In a second preferred embodiment, the separation of crude IPDA is carried out in a three column set-up, in which the first column K2-1 is a distillation column. The feed is preferably fed as a middle stream into column K2-1. In K2-1, fraction (iv) is preferably drawn-off at the top. A high boiling fraction comprising the components of fractions (i) to (iii) is preferably drawn-off from the sump of column K2-1. This fraction is introduced into a dividing wall column K2-2 in which fraction (i) is preferably drawn-off from the top, fraction (ii) is preferably drawn-off as a side stream and fraction (iii) is preferably drawn-off from the sump.

Fraction (iii) is preferably fed into a conventional distillation column K2-3 (IPDA recovery column), which is similar in function and design to the IPDA Recovery Column K1-2 in the embodiment according to FIG. 1. A schematic process flow diagram of this embodiment is illustrated in FIG. 2A.

Column K2-1 is preferably a conventional distillation column connected with a reboiler at the bottom and a condenser at the top of the column.

The column is preferably operated at:
a pressure in the range of 5 to 1000 mbar, preferably 7 to 500 mbar, more preferably 10 to 300 mbar;
a top temperature of preferably 10 to 120° C., more preferably 15 to 90° C., more even more preferably 20 to 75° C.;
a bottom temperature of preferably 90 to 280° C., more preferably 110 to 230° C., and even more preferably 120 to 210° C.

The number of theoretical trays is preferably 5 to 50, more preferably 7 to 40 and even more preferably 8 to 35.

The crude IPDA feed is preferably introduced into the middle of the column, preferably at a position at around 20 to 70% of the number of theoretical trays, more preferably 30 to 60% and even more preferably 40 to 55%.

Fraction (iv) is preferably withdrawn at the top of the column, where it is preferably condensed in a condenser W2-1, in one or two stages, which is operated at around 5 to 100, preferably 10 to 80, more preferably 15 to 60° C. The condensed phase is preferably fed to a phase separator F2-1 and preferably separated into a lighter organic phase (iv-a) and a heavier aqueous phase (iv-b). The lighter organic phase (iv-a) is preferably partially discarded and partially fed back to the column as reflux. The organic reflux ratio (reflux to organic distillate) is preferably in the range of 0.1 to 300, more preferably 0.5 to 280 and more preferably 1 to 50.

The bottom of the column is preferably connected to a reboiler W2-2 and a sump product comprising fractions (i) to (iii) is preferably withdrawn from the bottom of the column K2-1.

The reboiler is preferably a kettle type reboiler, a thermosyphon reboiler, a fired reboiler or a forced circulation reboiler, most preferably a kettle type reboiler or a thermosyphon reboiler. The sump product from column K2-1 is fed into the dividing wall column K2-1.

Column K2-2 is a dividing-wall column, preferably operated at:
a pressure in the range of 10 to 1000 mbar, preferably 30 to 700 mbar, more preferably 50 to 150 mbar;
a top temperature of preferably 100 to 280° C., more preferably 120 to 230° C., more even more preferably 140 to 170° C.;
a bottom temperature of preferably 150 to 300° C., more preferably 160 to 250° C., and even more preferably 170 to 200° C.

The feed from the sump of column K2-1 is preferably introduced into the middle of the column K2-2 in the one side of the dividing-wall, preferably at a position at around 10 to 70% of the number of theoretical trays, more preferably 20 to 60% and even more preferably 30 to 55%. A part of fraction (i) is preferably withdrawn at the top of the column, where it is preferably condensed in a condenser W2-3, which is operated at around 80 to 250, preferably 100 to 210, more preferably 120 to 150° C. Another part of fraction (i) is preferably refluxed to the column K2-2.

Fraction (ii) is preferably drawn-off at a side-take-off positioned at a position of around 30 to 80% of the number of theoretical trays, more preferably 40 to 70% and even more preferably 50 to 65%.

The bottom of the column K2-2—is preferably connected to a reboiler W2-4 and fraction (iii) is preferably withdrawn as a bottom product from the bottom of the column K2-2.

The reboiler W2-4 is preferably a kettle type reboiler, a thermosyphon reboiler, a fired reboiler or a forced circulation reboiler, most preferably a kettle type reboiler or a thermosyphon reboiler. The sump product, fraction (iii), from column K2-2 is fed into the IPDA recovery column K2-3. Column K2-3 in the embodiment according to FIG. 2A is operated and designed in a similar manner as the IPDA recovery column K1-2 according to the embodiment of FIG. 1.

Embodiment According to FIG. 2B:

A preferred variant of the embodiment of FIG. 2A is depicted in FIG. 2B.

In this variant, columns K2-1 and K2-2 are designed and operated in the same way, the equivalent columns K2-1 and K2-2 of the embodiment according to FIG. 2A are designed and operated.

From the sump of column K2-2, a fraction (iii) is removed, which comprises a higher IPNA mass fraction, a higher mass fraction of components having a higher boiling point than IPNA and IPDA.

Fraction (iii) is fed in a further distillation column K2-3 where it is further separated in a fraction (iii-1) comprising a higher mass fraction of cis-IPDA, compared to fraction (iii) and a fraction (iii-b) comprising a higher mass fraction of IPNA, compared to fraction (iii).

Fraction (iii-b) is then fed in a fourth column K2-4, which is operated as a conventional distillation column, where it is further separated into a fraction (iii-2) comprising a higher mass fraction of IPNA, compared to fraction (iii-b), into a fraction (iii-3) comprising a higher mass fraction of IPAA, compared to fraction (iii-b) and into a fraction (iii-4) comprising components having a higher boiling point than IPAA.

Column K2-3 is preferably operated at:
a pressure in the range of 5 to 300 mbar, preferably 15 to 100 mbar, more preferably 10 to 60 mbar;
a top temperature of preferably 80 to 210° C., more preferably 110 to 180° C., more even more preferably 120 to 170° C.;
a bottom temperature of preferably 120 to 270° C., more preferably 130 to 230° C., and even more preferably 140 to 210° C.

The number of theoretical trays is preferably 15 to 200, more preferably 20 to 100 and even more preferably 30 to 50.

Fraction (iii) from the sump of column K2-2 is preferably introduced into the middle of the column K2-3, preferably at a position at around 5 to 80% of the number of theoretical trays, more preferably 10 to 70% and even more preferably 20 to 50%.

Fraction (iii-1) is preferably withdrawn at the top of the column K2-3, where it is preferably condensed in a condenser W2-5, which is operated at around 40 to 190, preferably 80 to 180, more preferably 100 to 160° C. A part of fraction (iii-1) is preferably refluxed to the column K2-3.

The bottom of the column K2-3 is preferably connected to a reboiler W2-6 and fraction (iii-b) is preferably withdrawn as a bottom product from the bottom of the column K2-3.

The reboiler W2-6 is preferably a kettle type reboiler, a thermosyphon reboiler, a fired reboiler or a forced circulation reboiler, most preferably a kettle type reboiler or a thermosyphon reboiler. Fraction (iii-b) is introduced into column K2-4, preferably the middle of column K2-4.

Column K2-4 is preferably operated at:
a pressure in the range of 5 to 1000 mbar, preferably 10 to 500 mbar, more preferably 15 to 30 mbar;
a top temperature of preferably 110 to 280° C., more preferably 120 to 240° C., more even more preferably 130 to 150° C.;
a bottom temperature of preferably 150 to 340° C., more preferably 160 to 320° C., and even more preferably 170 to 210° C.

Fraction (iii-b) from the sump of column K2-3 is preferably introduced into the middle of the column K2-4, preferably at a position at around 10 to 90% of the number of theoretical trays.

Fraction (iii-2) is withdrawn at the top of the column K2-4, where it is preferably condensed in a condenser W2-7, which is operated at around 40 to 260, preferably 50 to 220, more preferably 60 to 130° C. A part of fraction (iii-2) is preferably refluxed to the column K2-4.

Fraction (iii-3) is withdrawn as a side draw.

The bottom of the column K2-3 is preferably connected to a reboiler W2-8 and fraction (iii-4) is preferably withdrawn as a bottom product from the bottom of the column K2-4.

The reboiler W2-8 is preferably a kettle type reboiler, a thermosyphon reboiler, a fired reboiler or a forced circulation reboiler, most preferably a kettle type reboiler or a thermosyphon reboiler. The embodiment according to FIG. 2B has the advantage that a fraction (iii-3) having a high content of the value product IPAA is obtainable.

Embodiment According to FIG. 2C:

A preferred variant of the embodiment of FIG. 2A is depicted in FIG. 2C.

In this variant, columns K2-1 and K2-2 are designed and operated in the same way, the equivalent columns K2-1 and K2-2 of the embodiment according to FIG. 2A, are designed and operated.

The variant according to FIG. 2C differs from the variant according to FIG. 2A in that 4 fractions are removed from column K2-3, instead of only two fractions being removed in the variant according to FIG. 2A.

In the embodiment according to FIG. 2C column K2-3 is preferably operated at:
a pressure in the range of 5 to 300 mbar, preferably 7 to 100 mbar, more preferably 10 to 60 mbar;
a top temperature of preferably 80 to 210° C., more preferably 110 to 180° C., more even more preferably 120 to 170° C.;
a bottom temperature of preferably 160 to 290° C., more preferably 170 to 250° C., and even more preferably 180 to 200° C.

The feed from the sump of column K2-2—fraction (iii)—is preferably introduced into the middle of the column K2-3, preferably at a position at around 5 to 80% of the number of theoretical trays, more preferably 10 to 70% and even more preferably 20 to 50%.

Fraction (iii-1) is preferably withdrawn at the top of the column K2-3, where it is preferably condensed in a condenser W2-5, which is operated at around 40 to 190, preferably 80 to 180, more preferably 100 to 160° C. A part of fraction (iii-1) is preferably refluxed to the column K2-4.

Fraction ((iii-2) is preferably withdrawn as a side draw above the feed stage.

Fraction ((iii-3) is preferably withdrawn as a side draw below the feed stage.

The bottom of the column K2-3—is preferably connected to a reboiler W2-6 and fraction ((iii-4) is preferably withdrawn as a bottom product from the bottom of the column K2-3.

The reboiler W2-6 is preferably a kettle type reboiler, a thermosyphon reboiler, a fired reboiler or a forced circulation reboiler, most preferably a kettle type reboiler or a thermosyphon reboiler. The embodiment according to FIG. 2C also has the advantage that a fraction ((iii-3) having a high content of the value product IPAA is obtainable.

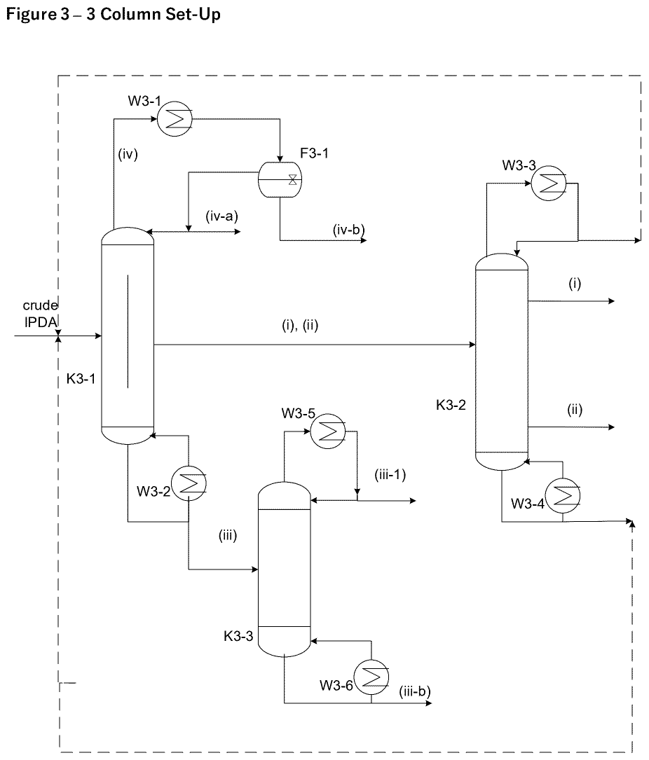

Embodiment According to FIG. 3:

In the embodiment according to FIG. 3, the separation of crude IPDA is carried out in a three column set-up, comprising a first dividing wall column K3-1 in which fraction (iv) is preferably draw-off from the top, fraction (iii) is preferably drawn-off from the sump and fractions (i) and (ii) are preferably jointly drawn-off as a side fraction. Fraction (iii) is fed to a conventional distillation column K3-3 (IPDA Recovery Column) where it is separated into fraction (iii-1), which is preferably obtained at the top of column and fraction (iii-b) which is preferably obtained at the sump of column K3-3.

The side draw from column K3-1 comprising fractions (ii) and (iii) is fed into a further conventional distillation column K3-2 where the feed stream is further separated into fraction (i), which is preferably delivered as a side-draw above the feed stage and into fraction (ii), which is preferably delivered as a side-draw below the feed stage. A schematic process flow diagram of this embodiment is illustrated in FIG. 3.

Column K3-1 is preferably operated at:
a pressure in the range of 10 to 1000 mbar, preferably 30 to 700 mbar, more preferably 50 to 150 mbar;
a top temperature of preferably 5 to 120° C., more preferably 10 to 100° C., more even more preferably 20 to 60° C.;
a bottom temperature of preferably 150 to 300° C., more preferably 160 to 250° C., and even more preferably 170 to 195° C.

The feed of crude IPDA is preferably introduced into the middle of the column, preferably at a position at around 20 to 70% of the number of theoretical trays, more preferably 30 to 60% and even more preferably 45 to 55%.

Fraction (iv) is preferably withdrawn at the top of the column, where it is preferably condensed in a condenser W3-1, which is operated at around 5 to 110, preferably 7 to 90, more preferably 10 to 50° C. The condensed phase is preferably fed to a phase separator F3-1 and preferably separated into a lighter organic phase (iv-a) and a heavier aqueous phase (iv-b). The lighter organic phase (iv-a) is preferably partially discarded and partially fed back to the column K3-1 as reflux.

Fractions (ii) and (iii) are preferably jointly withdrawn as a side-take-off in the middle section of column K3-1.

The bottom of the column K3-1 is preferably connected to a reboiler W3-2 and fraction (iii) is preferably withdrawn as a bottom product from the bottom of the column.

The side draw from column K3-1 comprising fractions (ii) and (iii) is fed into a further convention distillation column K3-2 where the feed stream is separated in fraction (i), which is preferably delivered as a side-draw above the feed stage in column K3-2 and a fraction (ii), which is preferably delivered as a side-draw below the feed stage.

In this embodiment, components with a boiling point lower than trans-IPDA are preferably removed at the top of column K3-2 and components with a boiling point lower than cis-IPDA are preferably removed as a sump product. Both streams, the top product and the sump product of column K3-2 can be recycled to the feed of column K3-1. A part of the top product from column K3-2 is preferably refluxed back to column K3-1.

The column K3-2 is preferably operated at:
a pressure in the range of 5 to 1000 mbar, preferably 10 to 500 mbar, more preferably 20 to 300 mbar;
a top temperature of preferably 90 to 240° C., more preferably 100 to 210° C., more even more preferably 120 to 190° C.;
a bottom temperature of preferably 110 to 270° C., more preferably 120 to 230° C., and even more preferably 140 to 210° C.

The feed from the side-take-off of column K3-1 is preferably introduced into the middle of column K3-2, preferably at a position at around 20 to 90% of the number of theoretical trays, more preferably 30 to 80% and even more preferably 40 to 70%.

The sump product from column K3-1 is fed into the IPDA recovery column K3-3. K3-3 in the embodiment according to FIG. 3 is preferably operated and designed in a similar manner as the IPDA Recovery Column according to the embodiment of FIG. 1.

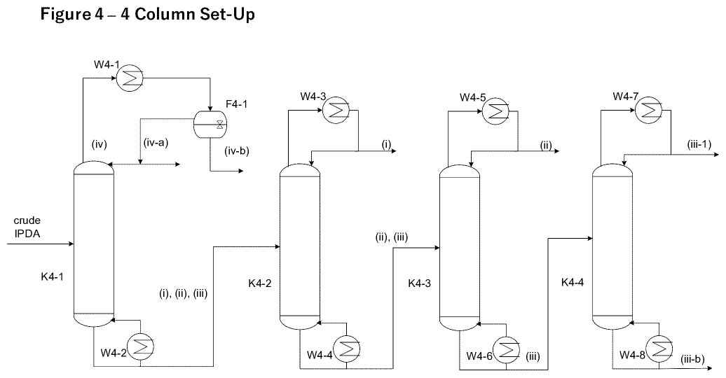

Embodiment According to FIG. 4:

In a fourth preferred embodiment, the separation of crude IPDA is carried out in a four column set-up, comprising a distillation column K4-1 in which fraction (iv) is preferably draw-off from the top and a fraction, comprising fractions (i) to (iii), is preferably drawn-off from the bottom. The bottom product, comprising fractions (i) to (iii) is fed into another conventional distillation column K4-2 where fraction (i) is preferably drawn-off as a top product and fractions (ii) and (iii) are preferably jointly drawn-off at the bottom. The bottom product from column K4-2 is fed into a third convention distillation column K4-3 in which fraction (ii) is preferably separated at the top and fraction (iii) is preferably obtained at the bottom. Fraction (iii) is again separated in a fourth conventional column K4-4 into fractions (iii-1) and (iii-b). A schematic process flow diagram of this embodiment is illustrated in FIG. 4.

The column K4-1 is preferably a conventional distillation column connected with a reboiler at the bottom and a condenser at the top of the column.

The column is preferably operated and designed in like column K2-1 described in the embodiment according to FIG. 2A.

The sump product from the column K4-1 is introduced into the column K4-2.

K4-2 is preferably a conventional distillation column connected with a reboiler at the bottom and a condenser at the top of the column.

The column K4-2 is preferably operated at:
a pressure in the range of 10 to 1000 mbar, preferably 50 to 700 mbar, more preferably 50 to 150 mbar;
a top temperature of preferably 100 to 260° C., more preferably 130 to 240° C., more even more preferably 140 to 190° C.;
a bottom temperature of preferably 130 to 280° C., more preferably 150 to 230° C., and even more preferably 170 to 200° C.

The feed to the column K4-2 is preferably introduced into the middle of the column, preferably at a position at around 10 to 90% of the number of theoretical trays, more preferably 20 to 80% and even more preferably 40 to 70%.

Fraction (i) is preferably partly withdrawn at the top of the column K4-2, where it is preferably condensed in a condenser W4-3, which is operated at around 40 to 240, preferably 50 to 220, more preferably 60 to 110° C. The other part of fraction (i) is preferably refluxed back into column K4-1.

The reflux ratio (reflux stream to fraction iii) is preferably in the range of 0.5 to 100, more preferably 1 to 30 and more preferably 5 to 10.

The bottom of the column is preferably connected to a reboiler W4-4.

The reboiler is preferably a kettle type reboiler, a thermosyphon reboiler, a fired reboiler or a forced circulation reboiler, most preferably a kettle type reboiler or a thermosyphon reboiler.

Fractions (ii) to (iii) are preferably withdrawn as a bottom product from the bottom of column K4-2.

The bottom's product from column K4-2 is preferably fed into the column K4-3.

Column K4-3 is preferably a conventional distillation column connected with a reboiler W4-6 at the bottom and a condenser W4-5 at the top of the column.

The column K4-3 is preferably operated at:
a pressure in the range of 10 to 1000 mbar, preferably 50 to 700 mbar, more preferably 50 to 150 mbar;
a top temperature of preferably 80 to 270° C., more preferably 120 to 240° C., more even more preferably 130 to 180° C.;
a bottom temperature of preferably 150 to 300° C., more preferably 170 to 250° C., and even more preferably 150 to 195° C.

The bottoms product of the column K4-2 is preferably introduced into the middle of the column K4-3, preferably at a position at around 30 to 95% of the number of theoretical trays, more preferably 40 to 90% and even more preferably 60 to 85%.

Fraction (ii) is preferably partly withdrawn at the top of the column, where it is preferably condensed in a condenser W4-5, which is operated at around 40 to 250, preferably 60 to 200, more preferably 80 to 160° C. The other part of fraction (ii) is preferably refluxed back to column K4-3. The reflux ratio (reflux to fraction (ii)) is preferably in the range of 0.8 to 50, more preferably 1 to 10 and more preferably 2 to 5.

The bottom of the K4-3 is preferably connected to a reboiler W4-6.

The reboiler is preferably a kettle type reboiler, a thermosyphon reboiler, a fired reboiler or a forced circulation reboiler, most preferably a kettle type reboiler or a thermosyphon reboiler.

Fraction (iii) is preferably withdrawn as a bottom product from the bottom of K4-3.

The bottom product from K4-3 is preferably fed into column K4-4. The column K4-4 in the embodiment according to FIG. 4 is operated and designed in a similar manner as the IPDA recovery column K1-2 according to the embodiment of FIG. 1.

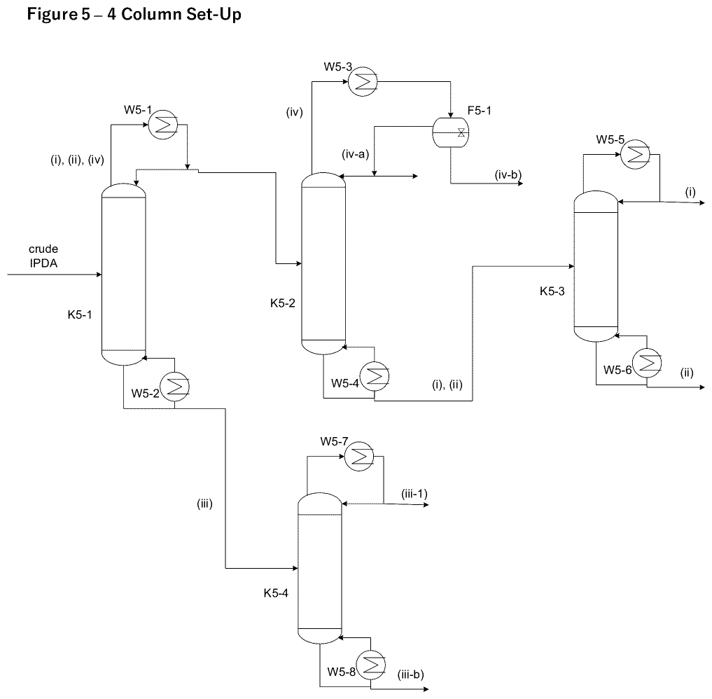

Embodiment According to FIG. 5:

In a fifth embodiment, the separation of crude IPDA is carried out in a another four column set-up. Crude IPDA is fed into a first conventional column K5-1 where fraction (iii) is preferably removed as a bottoms product and fractions (i), (ii) and (iv) are preferably jointly drawn-off at the top. The top product from column K5-1 is fed into a second conventional distillation column K5-2 in which fraction (iv) is preferably removed from the top and fractions (i) to (ii) are preferably jointly removed from the bottom. The bottom stream from column K5-2 is fed into a third convention column K5-3 where fraction (i) is preferably obtained at the top and fraction (ii) is preferably removed from the bottom. Fraction (iii) which is obtained as the bottom product from column K5-1 is fed into a fourth convention distillation column K5-4 and separated into fractions (iii-1) and (iii-b). A schematic process flow diagram of this embodiment is illustrated in FIG. 5.

Column K5-1 is preferably a conventional distillation column connected with a reboiler W5-2 at the bottom and a condenser W5-1 at the top of the column.

The column K5-1 is preferably operated at:
a pressure in the range of 10 to 1000 mbar, preferably 50 to 700 mbar, more preferably 50 to 150 mbar;
a top temperature of preferably 5 to 130° C., more preferably 30 to 110° C., more even more preferably 40 to 70° C.;
a bottom temperature of preferably 150 to 300° C., more preferably 170 to 250° C., and even more preferably 150 to 195° C.

Crude IPDA is preferably introduced into the middle of the column K5-1, preferably at a position at around 10 to 90% of the number of theoretical trays, more preferably 20 to 80% and even more preferably 30 to 70%.

The combined fractions (i), (ii) and (iv) are preferably partly withdrawn at the top of the column K5-1, where they are condensed in a condenser W5-1, which is operated at around 5 to 120, preferably 10 to 100, more preferably 30 to 60° C. The other part of the combined fractions (i), (ii) and (iv) is preferably refluxed back to column K5-1.

The bottom of the column K5-1 is preferably connected to a reboiler W5-2.

The reboiler is preferably a kettle type reboiler, a thermosyphon reboiler, a fired reboiler or a forced circulation reboiler, most preferably a kettle type reboiler or a thermosyphon reboiler.

Fraction (iii) is preferably withdrawn as a bottom product from the bottom of column K5-1. The top product from column K5-1 is introduced into the low boiler column K5-2.

Column K5-2 is preferably a conventional distillation column connected with a reboiler W5-4 at the bottom and a condenser W5-3 at the top of the column.

The column K5-2 is preferably operated at:
a pressure in the range of 50 to 1000 mbar, preferably 10 to 500 mbar, more preferably 15 to 300 mbar;
a top temperature of preferably 10 to 120° C., more preferably 15 to 90° C., more even more preferably 20 to 75° C.;
a bottom temperature of preferably 90 to 280° C., more preferably 110 to 230° C., and even more preferably 120 to 210° C.

The feed to column K5-2 is preferably introduced into the middle of the column, preferably at a position at around 10 to 90% of the number of theoretical trays, more preferably 20 to 80% and even more preferably 30 to 70%.

Fraction (iv) is preferably withdrawn at the top of the column, where it is preferably condensed in a condenser W5-3, which is operated at around 5 to 100, preferably 5 to 80, more preferably 5 to 60° C. The condensed phase is preferably fed to a phase separator F5-1 and preferably separated into a lighter organic phase (iv-a) and a heavier aqueous phase (iv-b). The lighter organic phase is preferably partially discarded and partially fed back to the column as reflux.

The bottom of the column K5-2 is preferably connected to a reboiler W5-4.

The reboiler is preferably a kettle type reboiler, a thermosyphon reboiler, a fired reboiler or a forced circulation reboiler, most preferably a kettle type reboiler or a thermosyphon reboiler.

Fractions (i) and (ii) are preferably jointly withdrawn as a bottoms product from the bottom of K5-2.

The sump product from K5-2 is preferably fed into the IPDA separation column K5-3.

The column K5-3 is preferably a conventional distillation column connected with a reboiler W5-6 at the bottom and a condenser W5-5 at the top of the column.

The column K5-3 is preferably operated at:
a pressure in the range of 5 to 1000 mbar, preferably 10 to 500 mbar, more preferably 20 to 300 mbar;
a top temperature of preferably 100 to 250° C., more preferably 110 to 220° C., more even more preferably 130 to 200° C.;
a bottom temperature of preferably 100 to 260° C., more preferably 110 to 220° C., and even more preferably 130 to 200° C.

The feed to column K5-3 is preferably introduced into the middle of the column, preferably at a position at around 10 to 90% of the number of theoretical trays, more preferably 20 to 80% and even more preferably 30 to 70%.

Fraction (i) is preferably withdrawn at the top of the column, where it is preferably condensed in a condenser W5-5, which is operated at around 40 to 230, preferably 50 to 200, more preferably 60 to 180° C. A part of fraction (ii) is preferably refluxed back to column K5-3.

The bottom of the column K5-3 is preferably connected to a reboiler W5-6.

The reboiler is preferably a kettle type reboiler, a thermosyphon reboiler, a fired reboiler or a forced circulation reboiler, most preferably a kettle type reboiler or a thermosyphon reboiler.

Fraction (ii) is preferably withdrawn as a bottoms product from the bottom of column K5-3.

The bottom product from the column K5-1 is preferably fed into the IPDA recovery column K5-4. The IPDA recovery column K5-4 in the embodiment according to this FIG. 4 is operated and designed in a similar manner as the IPDA recovery column K1-2 according to the embodiment of FIG. 1.

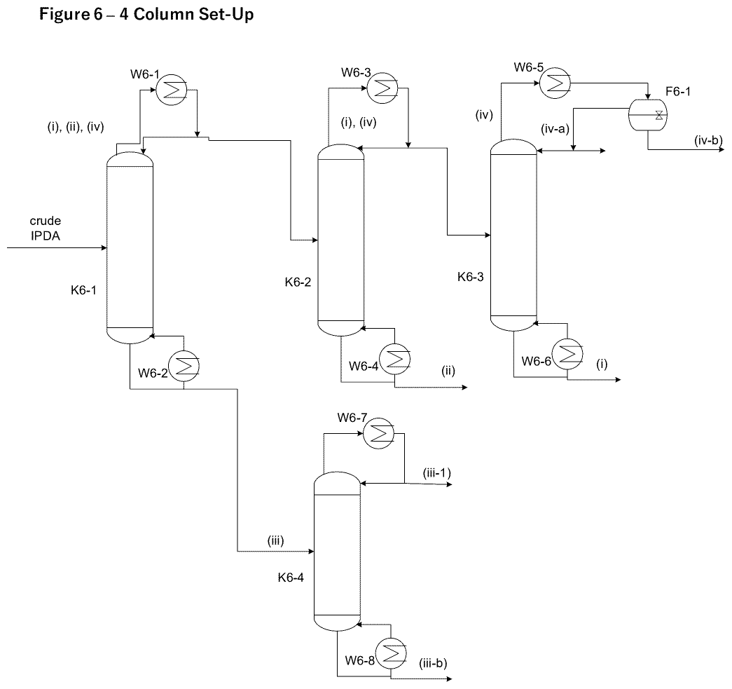

Embodiment According FIG. 6:

In a sixth embodiment, the separation of crude IPDA is carried out in a four-column set-up. Crude IPDA is fed into a first conventional column K6-1 (IPDA enrichment column) where fraction (iii) is preferably removed as a bottoms product and fractions (i), (ii) and (iv) are preferably jointly drawn-off at the top of column K6-1. The top product from K6-1 is fed into a second conventional distillation column K6-2 in which fractions (i) and (iv) are preferably jointly removed from the top and fraction (ii) is preferably removed from the bottom. The top product from K6-2 is fed into a third column K6-3 where the low boiling fraction (iv) is preferably removed as a top product and fraction (i) is preferably removed from the bottom. Fraction (iii), which is preferably obtained as the bottom product from the IPDA enrichment column K6-1 is further separated in a fourth conventional distillation column K6-4 (IPDA recovery column) to obtain fractions (iii-1) and (iii-b). A schematic process flow diagram of this embodiment is illustrated in FIG. 6.

The IPDA enrichment column K6-1 is preferably a conventional distillation column connected with a reboiler W6-2 at the bottom and a condenser W6-1 at the top of the column.

The column is preferably operated and designed in the same manner as the IPDA enrichment column K5-1 described in the embodiment according to FIG. 5.

The top product from column K6-1 is introduced to the column K6-2.

The column K6-2 is preferably a conventional distillation column connected with a reboiler W6-4 at the bottom and a condenser W6-3 at the top of the column.

The column K6-2 is preferably operated at:
a pressure in the range of 5 to 1000 mbar, preferably 10 to 500 mbar, more preferably 20 to 300 mbar;
a top temperature of preferably 80 to 230° C., more preferably 90 to 210° C., more even more preferably 110 to 200° C.;
a bottom temperature of preferably 100 to 260° C., more preferably 110 to 220° C., and even more preferably 130 to 200° C.

The feed to the K6-2 is preferably introduced into the middle of the column, preferably at a position at around 10 to 90% of the number of theoretical trays, more preferably 20 to 80% and even more preferably 30 to 70%.

Fractions (i) and (iv) are preferably jointly withdrawn at the top of the column, where they are condensed in a condenser W6-5, which is operated at around 40 to 210, preferably 50 to 190, more preferably 60 to 180° C. A part of fraction (i) and (iv) are preferably refluxed back to column K6-2.

The bottom of the column K6-2 is preferably connected to a reboiler W6-4.

The reboiler W6-4 is preferably a kettle type reboiler, a thermosyphon reboiler, a fired reboiler or a forced circulation reboiler, most preferably a kettle type reboiler or a thermosyphon reboiler.

Fraction (ii) is preferably withdrawn as a bottoms product from the bottom of K6-2.

The top product comprising fraction (i) and (iv) is fed into the column K6-3, which is preferably a conventional distillation column connected with a reboiler W6-6 at the bottom and a condenser W6-5 at the top of the column.

The column K6-3 is preferably operated at:
a pressure in the range of 10 to 1000 mbar, preferably 20 to 500 mbar, more preferably 30 to 200 mbar;
a top temperature of preferably 10 to 120° C., more preferably 15 to 90° C., more even more preferably 25 to 70° C.;
a bottom temperature of preferably 100 to 260° C., more preferably 110 to 220° C., and even more preferably 130 to 190° C.

The feed to K6-3 is preferably introduced into the middle of the column, preferably at a position at around 10 to 90% of the number of theoretical trays, more preferably 20 to 80% and even more preferably 30 to 70%.

Fraction (iv) is preferably withdrawn at the top of the column, where it is condensed in a condenser, W6-5 which is operated at around 5 to 100, preferably 10 to 80, more preferably 15 to 60° C. The condensed phase is preferably fed to a phase separator F6-1 and separated into a lighter organic phase (iv-a) and a heavier aqueous phase (iv-b). The lighter organic phase (iv-a) is preferably partially discarded and partially fed back to the column as reflux.

The bottom of the column K6-3 is preferably connected to a reboiler W6-6.

The reboiler W6-6 is preferably a kettle type reboiler, a thermosyphon reboiler, a fired reboiler or a forced circulation reboiler, most preferably a kettle type reboiler or a thermosyphon reboiler.

Fraction (i) is preferably withdrawn as a bottom product from the bottom of K6-3.

The sump product from the IPDA enrichment column K6-1 is fed into the IPDA recovery column K6-4. The IPDA recovery column K6-4 in the embodiment according to FIG. 6 is operated and designed in a similar manner as the IPDA recovery column K1-2 according to the embodiment of FIG. 1.

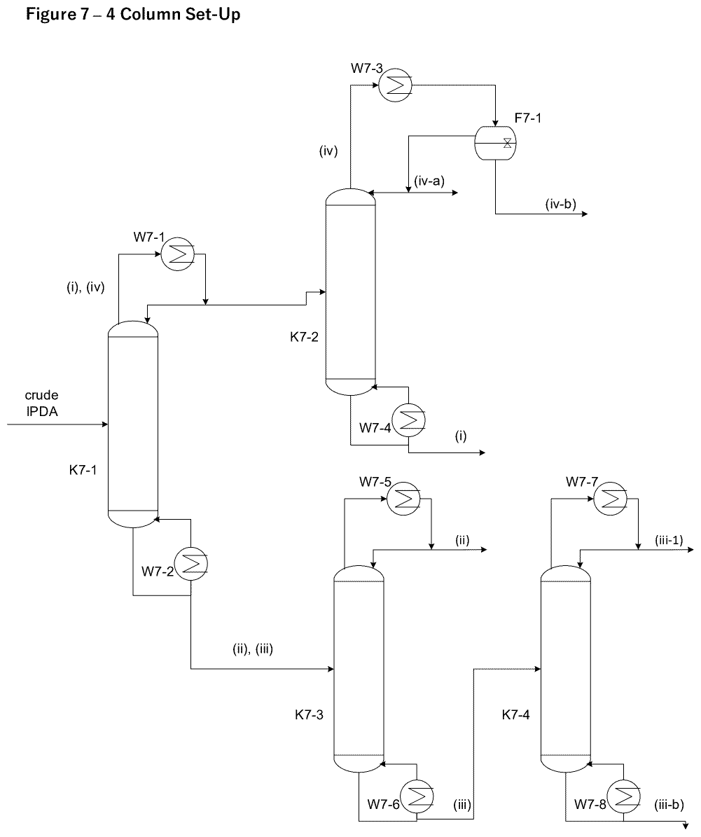

Embodiment According to FIG. 7:

In a seventh embodiment, the separation of crude IPDA is carried out in another four-column set-up. Crude IPDA is fed into a first conventional column K7-1 (IPDA cut-off column) where fractions (i) and (iv) are preferably jointly drawn-off at the top and fractions (ii) and (iii) are preferably jointly drawn-off from the bottom. The top product from K7-1 is fed into a second conventional K7-2 in which fraction (iv) is preferably removed from the top and fraction (i) is removed from the bottom. The bottom product of column K7-1 is fed into a third column K7-3, where fraction (ii) is preferably removed as a top product and fraction (iii) is preferably removed from the bottom. Fraction (iii), which is obtained as the bottom product from K7-3 is further separated in a fourth conventional distillation column K7-4 (IPDA recovery column) to obtain fractions (iii-1) and (iii-b). A schematic process flow diagram of this embodiment is illustrated in FIG. 7.

The IPDA cut-off column K7-1 is preferably a conventional distillation column connected with a reboiler W7-2 at the bottom and a condenser W7-1 at the top of the column.

The column K7-1 is preferably operated at:
a pressure in the range of 10 to 1000 mbar, preferably 50 to 700 mbar, more preferably 50 to 150 mbar;
a top temperature of preferably 90 to 260° C., more preferably 100 to 210° C., more even more preferably 120 to 180° C.;
a bottom temperature of preferably 130 to 290° C., more preferably 160 to 240° C., and even more preferably 130 to 190° C.

Crude IPDA is preferably introduced into the middle of the column K7-1, preferably at a position at around 10 to 90% of the number of theoretical trays, more preferably 20 to 80% and even more preferably 30 to 70%.

The combined fractions (i) and (iv) are preferably withdrawn at the top of the column K7-1, where they are condensed in a condenser W7-1, which is operated at around 20 to 240, preferably 30 to 190, more preferably 40 to 160° C. A part of the top product is preferably refluxed back to column K7-1.

The bottom of the column is preferably connected to a reboiler W7-2.

The reboiler W7-2 is preferably a kettle type reboiler, a thermosyphon reboiler, a fired reboiler or a forced circulation reboiler, most preferably a kettle type reboiler or a thermosyphon reboiler. The combined fractions (ii) and (iii) are preferably withdrawn as a sump product from the bottom of K7-1.

The top product from K7-1 is introduced to the column K7-2.

The column K7-2 is preferably a conventional distillation column connected with a reboiler W7-4 at the bottom and a condenser W7-3 at the top of the column.

The column K7-2 is preferably operated at:
a pressure in the range of 10 to 1000 mbar, preferably 10 to 500 mbar, more preferably 30 to 200 mbar;
a top temperature of preferably 10 to 120° C., more preferably 10 to 90° C., more even more preferably 25 to 70° C.;
a bottom temperature of preferably 100 to 260° C., more preferably 110 to 220° C., and even more preferably 130 to 190° C.

The feed to K7-2 is preferably introduced into the middle of the column, preferably at a position at around 10 to 90% of the number of theoretical trays, more preferably 20 to 80% and even more preferably 30 to 70%.

Fraction (iv) is preferably withdrawn at the top of the column, where it is condensed in a condenser W7-3, which is operated at around 5 to 100, preferably 7 to 80, more preferably 10 to 60° C. The condensed phase is preferably fed to a phase separator F7-1 and separated into a lighter organic phase (iv-a) and a heavier aqueous phase (iv-b). The lighter organic phase (iv-a) is preferably partially discarded and partially fed back to the column K7-2 as reflux.

The bottom of the column K7-2 is preferably connected to a reboiler W7-4.

The reboiler W7-4 is preferably a kettle type reboiler, a thermosyphon reboiler, a fired reboiler or a forced circulation reboiler, most preferably a kettle type reboiler or a thermosyphon reboiler. Fraction (i) is preferably withdrawn as a bottoms product from the bottom of K7-2.

The bottom product from K7-1 comprising fractions (ii) and (iii) is fed into the column K7-3, which is preferably a conventional distillation column connected with a reboiler W7-6 at the bottom and a condenser W7-5 at the top of the column.

The column K7-3 is preferably operated at:
a pressure in the range of 10 to 1000 mbar, preferably 50 to 700 mbar, more preferably 50 to 150 mbar;
a top temperature of preferably 100 to 260° C., more preferably 140 to 240° C., more even more preferably 150 to 190° C.;
a bottom temperature of preferably 150 to 300° C., more preferably 170 to 250° C., and even more preferably 175 to 195° C.

The feed to K7-3 is preferably introduced into the middle of the column, preferably at a position at around 10 to 90% of the number of theoretical trays, more preferably 20 to 80% and even more preferably 30 to 70%.

Fraction (ii) is preferably withdrawn at the top of the column, where it is preferably condensed in a condenser W7-5, which is operated at around 40 to 240, preferably 50 to 220, more preferably 60 to 170° C. A part of fraction (ii) is preferably refluxed back to column K7-3.

The bottom of the column K7-3 is preferably connected to a reboiler W7-6.

The reboiler W7-6 is preferably a kettle type reboiler, a thermosyphon reboiler, a fired reboiler or a forced circulation reboiler, most preferably a kettle type reboiler or a thermosyphon reboiler.

Fraction (iii) is preferably withdrawn as a bottoms product from the bottom of K7-3.

The bottoms product from K7-3 is preferably fed into the IPDA recovery column K7-4. The IPDA recovery column K7-4 in the embodiment according to FIG. 7 is operated and designed in a similar manner as the IPDA recovery column K1-2 according to the embodiment of FIG. 1.

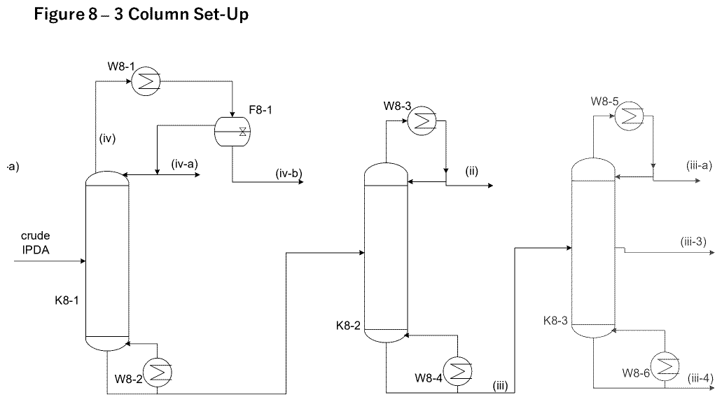

Embodiment According to FIG. 8:

In an eight embodiment, the separation of crude IPDA is carried out in a three-column set-up. Crude IPDA is fed into a first conventional column K8-1 (low boiler column) where fraction (iv) is preferably drawn-off at the top of K8-1 and fractions (ii) and (iii) are preferably jointly drawn-off from the bottom.

The bottom product of column K8-1 is fed into a second column K8-2 (IPDA column), where fraction (ii) is preferably removed as a top product and fraction (iii) is preferably removed from the bottom.

Fraction (iii), which is obtained as the bottom product from K8-2 is further separated in a third conventional distillation column K8-3 (IPAA recovery column) to obtain a fraction (iii-a) as a top product, a fraction (iii-3) as a side-draw and fraction (iii-4) as the bottom products.

This embodiment is especially preferred if the CTR of the crude IPDA is 70:30 or more, preferably 73:27 or more, more preferably 75:25 or more and even more preferably 80:20 or more. If the CTR of raw IPDA is in the present range, it may be possible to carry out the distillation in column K8-2 in a manner that an additional fraction (i) must not be separated in order to obtain a fraction (ii) with a sufficiently high CTR required for applications requiring IPDA with a high CTR. It is further possible to carry out the distillation in column K8-3 in a manner that is possible so that the ratio of fraction (iii-2) to fraction (iii-1) in fraction (iii-a) in column K8-3 is 90:10 or more, preferably 95:5 or more and more preferably 99:1 or more. In this manner little or no IPDA is lost in fraction (iii-a), enabling an overall high IPDA recovery rate.

A schematic process flow diagram of this embodiment is illustrated in FIG. 8.

The column K8-1 is preferably a conventional distillation column connected with a reboiler W8-2 at the bottom and a condenser W8-1 at the top of the column.

The column is preferably operated at:
a pressure in the range of 5 to 1000 mbar, preferably 7 to 500 mbar, more preferably 10 to 300 mbar;
a top temperature of preferably 10 to 120° C., more preferably 20 to 100° C., more even more preferably 30 to 90° C.;
a bottom temperature of preferably 90 to 280° C., more preferably 110 to 230° C., and even more preferably 120 to 210° C.

The number of theoretical trays is preferably 5 to 50, more preferably 7 to 40 and even more preferably 8 to 35.

The crude IPDA feed is preferably introduced into the middle of the column, preferably at a position at around 20 to 70% of the number of theoretical trays, more preferably 30 to 60% and even more preferably 40 to 55%.

Fraction (iv) is preferably withdrawn at the top of the column, where it is preferably condensed in a condenser W8-1, in one or two stages, which is operated at around 5 to 100, preferably 10 to 80, more preferably 15 to 60° C. The condensed phase is preferably fed to a phase separator F8-1 and preferably separated into a lighter organic phase (iv-a) and a heavier aqueous phase (iv-b). The lighter organic phase (iv-a) is preferably partially discarded and partially fed back to the column as reflux. The organic reflux ratio (reflux to organic distillate) is preferably in the range of 0.1 to 300, more preferably 0.5 to 280 and more preferably 1 to 50.

The bottom of the column is preferably connected to a reboiler W2-2 and a fraction comprising fractions (ii) and (iii) is preferably withdrawn from the bottom of the column K8-1.

The reboiler is preferably a kettle type reboiler, a thermosyphon reboiler, a fired reboiler or a forced circulation reboiler, most preferably a kettle type reboiler or a thermosyphon reboiler.

The sump product from column K8-1 is fed into column K8-2 (IPDA column).

The column K8-2 is preferably operated at:
a pressure in the range of 10 to 1000 mbar, preferably 10 to 500 mbar, more preferably 30 to 200 mbar;
a top temperature of preferably 100 to 240° C., more preferably 120 to 200° C., more even more preferably 140 to 180° C.;
a bottom temperature of preferably 120 to 260° C., more preferably 140 to 220° C., and even more preferably 150 to 200° C.

The feed to K8-2 is preferably introduced into the middle of the column, preferably at a position at around 10 to 90% of the number of theoretical trays, more preferably 20 to 80% and even more preferably 30 to 70%.

Fraction (ii) is preferably withdrawn at the top of the column, where it is preferably condensed in a condenser W8-3, which is operated at around 40 to 240, preferably 60 to 220, more preferably 80 to 200° C. A part of fraction (ii) is preferably refluxed back to column K8-2.

The bottom of the column K8-2 is preferably connected to a reboiler W8-4.

The reboiler W8-4 is preferably a kettle type reboiler, a thermosyphon reboiler, a fired reboiler or a forced circulation reboiler, most preferably a kettle type reboiler or a thermosyphon reboiler.

Fraction (iii) is preferably withdrawn as a bottoms product from the bottom of K8-2.

The feed from column K8-2 to column K8-3 is preferably introduced into the middle of the column, preferably at a position at around 10 to 90% of the number of theoretical trays, more preferably 20 to 80% and even more preferably 30 to 70%.

The pressure in column K8-3 is usually in the range of 1 to 500 mbar, preferably 5 to 100 mbar, more preferably 10 to 50 mbar; and the top temperature is preferably in the range of 80 to 220° C., more preferably 90 to 200° C., more even more preferably 110 to 150° C.; and the bottom temperature preferably is preferably in the range of 100 to 270° C., more preferably 140 to 225° C., and even more preferably 150 to 210° C.

Fraction (iii-a) is preferably withdrawn at the top of the column K8-3, where it is preferably condensed in a condenser W8-5, which is operated at around 40 to 240, preferably 50 to 220, more preferably 60 to 170° C. A part of fraction (iii-a) is preferably refluxed back to column K8-3.

The bottom of the column K8-3 is preferably connected to a reboiler W8-6.

The reboiler W8-6 is preferably a kettle type reboiler, a thermosyphon reboiler, a fired reboiler or a forced circulation reboiler, most preferably a kettle type reboiler or a thermosyphon reboiler.

Fraction (iii-4) is preferably withdrawn as a bottoms product from the bottom of K8-3.

An additional fraction (iii-3) is withdrawn as a side product from column K8-3.

The invention is not limited to the aforementioned, specific embodiments. These embodiments are merely understood to exemplify the principle of the invention. Further embodiments in which dividing wall columns and/or conventional columns are interconnected and where the fractions are drawn-off at different parts of the respective columns to obtain the desired fractions are considered to be included under the general inventive concept of this invention.

In particular the invention comprises embodiments wherein (A) the fraction (iii-b) is further separated in an additional column into a fraction ((iii-2) comprising a higher mass fraction of IPNA compared to fraction (iii); a fraction ((iii-3) comprising a higher mass fraction of isophorone amino alcohol (IPAA) compared to fraction (iii); and a fraction ((iii-4) comprising a higher mass fraction of components having a boiling point higher than IPAA compared to fraction (iii) or wherein (B) instead of a fraction (iii-b), three fractions ((iii-2), ((iii-3) and ((iii-4) are separated in the last column of the respective multi-column-setup without an additional column.

The fractions which are obtained according to the process of the invention preferably have the following composition:

### Fraction (iv):

Water: 10 to 60 percent by weight, preferably 20 to 50 percent by weight;
Organics with a boiling point lower than trans IPDA: 40 to 90 percent by weight, preferably 50 to 80 percent by weight;
   Fraction (iv-a):
Organics with a boiling point lower than trans IPDA: 69.5 to 90 percent by weight, preferably 75 to 85 percent by weight;
Water: 10 to 30 percent by weight, preferably 12 to 16 percent by weight,
IPDA: 0 to 0.5 percent by weight.
   Fraction (iv-b):
Water: 95 to 100, preferably 96 to 99 percent by weight;
IPDA: 0 to 0.1, preferably 0 to 0.001 percent by weight,
Organics with a boiling point lower than trans-IPDA: 0 to 4.9, preferably 0 to 3.999 percent by weight;

### Fraction (i):

Trans-IPDA: 29.5 to 50, preferably 34.5 to 45 percent by weight;
Cis-IPDA: 50 to 70, preferably 55 to 65 percent by weight;
Others: 0 to 0.5 percent by weight.

### Fraction (ii):

Trans-IPDA: 19.5 to 30, preferably 21.5 to 28 percent by weight;
Cis-IPDA: 70 to 80, preferably 68 to 78 percent by weight;
Others: 0 to 0.5 percent by weight
   Fraction (iii):
Trans-IPDA: 2 to 8, preferably 4.5 to 5 percent by weight;
Cis-IPDA: 10 to 50, preferably 30 to 40 percent by weight;
IPNA: 0 to 5, preferably 0.2 to 0.5 percent by weight;
IPAA: 5 to 35, preferably 10 to 25 percent by weight;
Others: 0 to 50 percent by weight, preferably 0 to 30 percent by weight.
   Fraction (iii-1):
Trans IPDA: 20 to 40 percent by weight;
Cis IDPA: 60 to 79.9 percent by weight;
Others: 0-0.1 percent by weight
   Fraction (iii-b):
IPNA: 0.1 to 10 percent by weight;
IPAA: 10 to 40 percent by weight;
Components having a higher boiling point than IPAA: 50 to 90 percent by weight

In the special embodiment where an additional fractions ((iii-2), ((iii-3) and ((iii-4) are obtained, the composition of the fractions ((iii-2), ((iii-3) and ((iii-4) are preferably as follows:

Fraction ((iii-2):
IPNA: 90 to 100 percent by weight;
IPAA: 0 to 10 percent by weight.
   Fraction ((iii-3):
IPAA: 80 to 100, preferably 95 to 100 percent by weight;
Components with a higher boiling point than IPAA: 0 to 20 percent by weight, preferably 0 to 5 percent by weight.
   Fraction ((iii-4):
Components with a boiling point higher than IPAA: 100 percent by weight.

The IPNA mass fraction in fractions (i) and (ii) is preferably 0.2 percent by weight or less, more preferably 0.1 percent by weight or less and most preferably 0.05 percent by weight or less.

Surprisingly, it was found that the IPDA-fractions (ii) and (iii) prepared by the inventive process lead to improved properties in down-stream applications, which are probably attributable to the depletion of IPNA.

The process according to the present invention allows a high IPDA recovery, even if the IPDA yield in the crude IPDA decreases over time due to a reduction of catalyst activity or selectivity, which may occur over prolonged operating periods. In particular, the process of the present invention allows the conversion of IPN to IPDA without a post-hydrogenation reactor which is often required to decrease the yield of undesired IPNA. For higher IPDA recovery rates the specific energy demand per tonne of product further decreases and contributes to the reduction of the carbon footprint of the IPDA production process. The process according to the present invention results to a lower depletion of raw materials.

The process of the present invention also allows for the recovery of IPAA from crude IPDA. IPAA is a value product which is useful in several applications.

A process according to the invention is demonstrated by the following examples:

### EXAMPLES

The examples are based on calculations performed using a process simulation model.

The simulations have been performed using CHEMASIM®. For the calculation of thermodynamic properties of pure components, like the vapor pressure, DIPPR correlations have been used. For the description of phase equilibria, the ideal gas law is used to describe the vapor phase and the NRTL excess Gibbs energy model is used for the description of the liquid phase. The parameters of the DIPPR correlations and the parameters of the NRTL model were adjusted to experimental data. For the components, for which no experimental data are available, the UNIFAC group contribution method was used for the description of the liquid phase in phase equilibria calculations. The distillation columns have been modelled and calculated using the equilibrium stage model. The employed simulation and thermodynamic property models have been adjusted to reproduce experimental and plant data with very good accuracy.

The composition of the crude IPDA feed streams were set at the compositions listed in Table 1 below:

TABLE 1
Composition of the crude IPDA feed stream

| (in wt. %) | Feed 1 | Feed 2 | Feed 3 |
|---|---|---|---|
| IPDA | 81.50 | 88.50 | 88.50 |
| Cis-Trans-ratio of IPDA | 2.50 | 2.50 | 3.10 |
| (CTR) | | | |
| H2O | 9.00 | 9.00 | 9.10 |
| IPNA | 0.08 | 0.08 | 0.08 |
| IPAA | 0.70 | 0.70 | 0.70 |
| Low boilers: components | 3.40 | 1.40 | 1.40 |
| with a boiling point lower | | | |
| than trans-IPDA | | | |
| High boilers: components | 5.40 | 0.35 | 0.35 |
| with a boiling point higher | | | |
| IPAA | | | |

Process parameters that met following product and process specifications were determined for the respective configuration under investigation using the simulation model:
Fraction (i): Trans-IPDA composition equal to 43.2 percent by weight
Fraction (ii): Cis-IPDA composition higher or equal to 75.5 percent by weight
Fraction (iii-1): Cis-IPDA composition higher or equal to 75.5 percent by weight, so that fraction (iii-1) can be mixed further either with fraction (i) or fraction (ii)
Fractions (i), (ii), (iii-1) and their mixtures: Total IPDA composition equal to or higher than 99.8 percent by weight.
Fractions (i), (ii), (iii-1) and their mixtures: Total IPNA composition lower than or equal to 500 ppm by weight.
Fractions (i), (ii), (iii-1) and their mixtures: Total composition of all other heavy boiling components with a boiling point higher than IPAA less or equal to 250 ppm by weight.
Fractions (i), (ii), (iii-1) and their mixtures: H₂O composition lower or equal to 250 ppm by weight.
Fraction ((iii-3): IPAA composition equal to 96.5 percent by weight.
The fractions (iii), (iii-b) and ((iii-4) in the sump of the corresponding distillation columns are allowed to reach a maximum temperature of 185° C., to prevent thermal degradation.

When the process parameters were determined which met the aforementioned specification, the following key-performance indices (KPIs) were determined in order to compare the examples with each other:

$$Specific energy demand = {Q}_{reboiler} / ( {m}_i + {m}_ii ) in kW / t$$

Product loss IPDA: cis- and trans-IPDA in fraction (iii) in kg/kg related to the quantity of IPDA in the crude IPDA stream
Product loss IPAA: IPAA in fraction ((iii-4) in kg/kg related to the quantity of IPAA in crude IPDA.

### Example 1 and Comparative Example 1

In Example 1, a process configuration according to FIG. 1 was calculated.

Comparative Example 1 used the configuration according to FIG. 1, but without the IPDA-recovery column K1-2.

The configuration of the dividing wall column K1-1 and the column K1-2 is depicted in Table 2 and Table 3:

TABLE 2
IPDA dividing wall column (K1-1)

| | Feed 2 |
|---|---|
| Theoretical stages HK | 90 |
| Theoretical stages VK | 40 |
| Feed stage | 20 |
| Stage SA FR_3 | 60 |
| Stage SA FR_4 | 28 |
| TWU | 26 |
| P condenser (bar) | 0.100 |
| T condenser (° C.) | 45 |
| T head (° C.) | 157 |
| T side draw FR_3 (° C.) | 167.8 |
| T side draw FR_4 (° C.) | 168.3 |
| T bottoms (° C.) | 176 |
| T reboiler (° C.) | 182 |
| liquid split above dividing | 0.5 |
| wall | |
| gas split bellow dividing | 0.7 |
| wall | |
| Reflux ratio (org.) | 225.5 |
| Heat duty reboiler (kW) | 1449 |

TABLE 3
IPDA recovery column (K1-2)

| | Feed 2 |
|---|---|
| Theoretical stages (feed | 40 |
| stage) | |
| Feed stage | 5 |
| P condenser (bar) | 0.035 |
| T condenser (° C.) | 141 |
| T head (° C.) | 141 |
| T bottoms (° C.) | 166 |
| T reboiler (° C.) | 167 |
| Reflux ratio | 6.5 |
| Heat duty reboiler (kW) | 28 |

A crude IPDA feed stream having the composition designated as Feed 2 in Table 1 was used.

In Example 1, the specific energy demand was 774 kW/t_{IPDA}.

In Comparative Example 1, the specific energy demand was slightly higher at 777 kW/t_{IPDA}.

However, the IPDA loss in the inventive Example 1 could be reduced by 96% from 2.4% IPDA loss in Comparative Example 1 to 0.1% in the configuration in Example 1.

Surprisingly, using a configuration according to Example 1 was able to reduce the IPDA-loss significantly compared to using a configuration according to Comparative Example 1, while it was even possible to slightly reduce the specific energy demand in the distillation process. If the energy demand for the disposal of the sump stream of column K1-2 in the Comparative Example 1 is taken into account, the advantage of the configuration in Example 1 is even higher, regarding the specific energy demand per tonne of IPDA product.

### Example 2 and Comparative Example 2

In Example 2, a process configuration according to FIG. 2A was calculated.

Comparative Example 2 used the configuration according to FIG. 2A, but without the IPDA recovery column K2-3.

In Example 2a and Comparative Example 2a, a crude IPDA feed stream having the composition designated as Feed 1 in Table 1 was used.

In Example 2b and Comparative Example 2b, a crude IPDA feed stream having the composition designated as Feed 2 in Table 1 was used.

The configuration of the columns is depicted in Table 4, 5 and 6.

TABLE 4
low boiler column K2-1

| | Feed 1 | Feed 2 |
|---|---|---|
| Theoretical stages (feed stage) | 32 | 32 |
| Feed stage | | |
| P condenser (bar) | 0.100 | 0.100 |
| T condenser (° C.) | 45 | 45 |
| T head (° C.) | 78 | 74 |
| T bottoms (° C.) | 169 | 168 |
| T reboiler (° C.) | 170 | 168 |
| Reflux ratio org. (g/g) | 1.6 | 1.6 |
| Heat duty reboiler (kW) | 308 | 296 |

TABLE 5
IPDA dividing wall column K2-2

| | Feed 1 | Feed 2 |
|---|---|---|
| Theoretical stages HK | 67 | 67 |
| Theoretical stages VK | 24 | 24 |
| Feed stage | 10 | 10 |
| Stage SA | 38 | 38 |
| P condenser (bar) | 0.050 | 0.050 |
| T condenser (° C.) | 113 | 113 |
| T head (° C.) | 149 | 149 |
| T side draw (° C.) | 149.3 | 149.3 |
| T bottoms (° C.) | 159 | 152 |
| T reboiler (° C.) | 161 | 153 |
| liquid split above dividing wall | 0.7 | 0.7 |
| gas split bellow dividing wall | 0.5 | 0.5 |
| Reflux ratio | 7.2 | 7.2 |
| Heat duty reboiler (kW) | 418 | 442 |

TABLE 6
IPDA recovery column K2-3

| | Feed 1 | Feed 2 |
|---|---|---|
| Theoretical stages (feed stage) | 46 | 46 |
| Feed stage | 10 | 10 |
| P condenser (bar) | 0.035 | 0.035 |
| T condenser (° C.) | 140 | 140 |
| T head (° C.) | 141 | 141 |
| T bottoms (° C.) | 184 | 166 |
| T reboiler (° C.) | 204 | 172 |
| Reflux ratio | 3.8 | 4.7 |
| Heat duty reboiler (kW) | 51 | 57 |

In Example 2a, the specific energy demand was 553 kW/t_{IPDA}.

In Comparative Example 2a, the specific energy demand was slightly higher at 559 kW/t_{IPDA}.

In Example 2b, the specific energy demand was 499 kW/t_{IPDA}.

In Comparative Example 2a, the specific energy demand was comparable at 499 kW/t_{IPDA}.

However, the IPDA loss in the inventive Example 2a could be reduced by 85%, from 5.7% IPDA loss in Comparative Example 2a to 0.9% in the inventive Example 2a.

Also, the IPDA loss in the inventive Example 2b could be reduced by 99%, from 7.0% IPDA loss in Comparative Example 2a to 0.1% in the inventive Example 2b.

Surprisingly, using a configuration according to Examples 2a and 2b was able to reduce the IPDA-loss significantly compared to using a configuration according to Comparative Examples 2a and 2b, while it was even possible to slightly reduce the specific energy demand in the distillation process. If the energy demand for the disposal of the sump stream of column K2-2 in the Comparative Examples 2a and 2b is taken into account, the advantage of the configuration in the inventive Examples 2a and 2b is even higher, regarding the specific energy demand per tonne of IPDA product.

### Example 3

In Example 3, a configuration according to FIG. 2A was used.

A crude IPDA feed stream having the composition designated as Feed 3 in Table 1 was used.

The configuration of the columns is depicted in Table 7, 8 and 9.

TABLE 7
low boiler column K2-1

| | Feed 3 |
|---|---|
| Theoretical stages (feed stage) | 32 |
| Feed stage | |
| P condenser (bar) | 0.100 |
| T condenser (° C.) | 45 |
| T head (° C.) | 74 |
| T bottoms (° C.) | 169 |
| T reboiler (° C.) | 169 |
| Reflux ratio org. (g/g) | 1.6 |
| Heat duty reboiler (kW) | 296 |

TABLE 8
Dividing wall column K2-2

| | Feed 3 |
|---|---|
| Theoretical stages HK | 67 |
| Theoretical stages VK | 24 |
| Feed stage | 10 |
| Stage SA | 35 |
| P condenser (bar) | 0.050 |
| T condenser (° C.) | 113 |
| T head (° C.) | 149 |
| T side draw (° C.) | 149.4 |
| T bottoms (° C.) | 152 |
| T reboiler (° C.) | 152 |
| liquid split above dividing wall | 0.7 |
| gas split bellow dividing wall | 0.5 |
| Reflux ratio | 10.6 |
| Heat duty reboiler (kW) | 551 |

TABLE 9
Dividing wall column K2-2

| | Feed 3 |
|---|---|
| Theoretical stages (feed stage) | 46 |
| Feed stage | 25 |
| P condenser (bar) | 0.035 |
| T condenser (° C.) | 141 |
| T head (° C.) | 141 |
| T bottoms (° C.) | 168 |
| T reboiler (° C.) | 141 |
| Reflux ratio | 4.7 |
| Heat duty reboiler (kW) | 69 |

The specific energy demand was 479 kW/t_{IPDA}. The IPDA loss was only 0.01%. Due to the configuration, IPAA present in fraction (iii-b) was not recovered (IPDA recovery=0%).

### Example 4

In Example 4, a configuration according to FIG. 2C was calculated.

A crude IPDA feed stream having the composition designated as Feed 3 in Table 1 was used.

Example 4 differs from Example 3 therein, that instead of a fraction (iii-b) three other fractions ((iii-2), ((iii-3) and ((iii-4) were separated in the IPDA recovery column K2-3.

The configuration of the columns is depicted in Table 10, 11 and 12.

TABLE 10
Low boiler column K2-1

| | Feed 3 (with IPAA recovery) |
|---|---|
| Theoretical stages (feed stage) | 32 |
| Feed stage | |
| P condenser (bar) | 0.100 |
| T condenser (° C.) | 45 |
| T head (° C.) | 74 |
| T bottoms (° C.) | 169 |
| T reboiler (° C.) | 169 |
| Reflux ratio org. (g/g) | 1.6 |
| Heat duty reboiler (kW) | 296 |

TABLE 11
Dividing wall column K2-2

| | Feed 3 (with IPAA recovery) |
|---|---|
| Theoretical stages HK | 67 |
| Theoretical stages VK | 24 |
| Feed stage | 10 |
| Stage SA | 35 |
| P condenser (bar) | 0.050 |
| T condenser (° C.) | 113 |
| T head (° C.) | 149 |
| T side draw (° C.) | 149.4 |
| T bottoms (° C.) | 152 |
| T reboiler (° C.) | 152 |
| liquid split above dividing wall | 0.7 |
| gas split bellow dividing wall | 0.5 |
| Reflux ratio | 10.6 |
| Heat duty reboiler (kW) | 551 |

TABLE 12
IPDA recovery column K2-2 with IPAA recovery:

| | Feed 3 (with IPAA recovery) |
|---|---|
| Theoretical stages (feed stage) | 46 |
| Feed stage | 25 |
| P condenser (bar) | 0.035 |
| T condenser (° C.) | 141 |
| T head (° C.) | 141 |
| T bottoms (° C.) | 185 |
| T reboiler (° C.) | 141 |
| Reflux ratio | 4.6 |
| Heat duty reboiler (kW) | 63 |

This configuration allowed the recovery of an IPAA fraction ((iii-3). The IPAA recovery from the crude IPDA feed was 85.9%.

### Comparative Examples 5a and 5b

In Comparative Example 5, a configuration according to FIG. 4 was used, but without IPDA recovery column K4-4.

In Comparative Example 5a, a crude IPDA feed stream having the composition designated as Feed 1 in Table 1 was used.

In Comparative Example 5b, a crude IPDA feed stream having the composition designated as Feed 2 in Table 1 was used.

The configuration of the columns is depicted in Table 13, 14 and 15

TABLE 13
Low boiler column K4-1

| | Feed 1 | Feed 2 |
|---|---|---|
| Theoretical stages (feed stage) | 32 | 32 |
| Feed stage | | |
| P condenser (bar) | 0.100 | 0.100 |
| T condenser (° C.) | 45 | 45 |
| T head (° C.) | 76 | 72 |
| T bottoms (° C.) | 169 | 168 |
| T reboiler (° C.) | 170 | 168 |
| Reflux ratio org. (g/g) | 4.4 | 9.3 |
| Heat duty reboiler (kW) | 295 | 284 |

TABLE 14
IPDA separation column K4-2

| | Feed 1 | Feed 2 |
|---|---|---|
| Theoretical stages (feed stage) | 30 | 30 |
| Feed stage | 20 | 20 |
| P condenser (bar) | 0.080 | 0.080 |
| T condenser (° C.) | 161 | 161 |
| T head (° C.) | 161 | 161 |
| T bottoms (° C.) | 163 | 162 |
| T reboiler (° C.) | 164 | 162 |
| Reflux ratio | 8.4 | 8.4 |
| Heat duty reboiler (kW) | 355 | 378 |

TABLE 14
Low boiler column K4-3

| | Feed 1 | Feed 2 |
|---|---|---|
| Theoretical stages (feed stage) | 19 | 19 |
| Feed stage | 16 | 16 |
| P condenser (bar) | 0.050 | 0.050 |
| T condenser (° C.) | 149 | 149 |
| T head (° C.) | 149 | 149 |
| T bottoms (° C.) | 168 | 155 |
| T reboiler (° C.) | 176 | 156 |
| Reflux ratio | 3.0 | 2.6 |
| Heat duty reboiler (kW) | 426 | 422 |

In Comparative Example 5a, the specific energy demand was 635 kW/t_{IPDA}.

In Comparative Example 5b, the specific energy demand was slightly lower at 587 kW/t_{IPDA}.

In Comparative Example 5a, the IPDA loss was 3.8%, whereas in Comparative Example 5a, the IPDA loss was 3.3%.

### Example 6 and Comparative Example 6

In Example 6, a configuration according to FIG. 8 was used and in Comparative Example 6, a configuration according to FIG. 8 was used, but without IPDA recovery column K8-3

In both examples, a feed having the composition designated as Feed 3 in Table 1 was used.

The configuration of the columns is depicted in Table 16, 17 and 18:

TABLE 16
Low boiler column K8-1:

| | Comparative Example 6 | Example 6 |
|---|---|---|
| Theoretical stages (feed stage) | 32 | 32 |
| Feed stage | 11 | 11 |
| P condenser (bar) | 0.100 | 0.100 |
| T condenser (° C.) | 45 | 45 |
| T head (° C.) | 72 | 72 |
| T bottoms (° C.) | 169 | 169 |
| T reboiler (° C.) | 169 | 169 |
| Reflux ratio org. (g/g) | 9.3 | 9.3 |
| Heat duty reboiler (kW) | 284.6 | 284.6 |

TABLE 17
IPDA column K8-2:

| | Comparative Example 6 | Example 6 |
|---|---|---|
| Theoretical stages (feed stage) | 28 | 28 |
| Feed stage | 24 | 24 |
| P condenser (bar) | 0.080 | 0.080 |
| T condenser (° C.) | 162 | 162 |
| T head (° C.) | 162 | 162 |
| T bottoms (° C.) | 183 | 183 |
| T reboiler (° C.) | 185 | 185 |
| Reflux ratio org. (g/g) | 1.8 | 1.8 |
| Heat duty reboiler (kW) | 444 | 444 |

TABLE 18
IPDA recovery column K8-3 with IPAA recovery:

| | Comparative Example 6 | Example 6 |
|---|---|---|
| Theoretical stages (feed stage) | n.a. | 30 |
| Feed stage | n.a. | 22 |
| P condenser (bar) | n.a. | 0.025 |
| T condenser (° C.) | n.a. | 133 |
| T head (° C.) | n.a. | 134 |
| T bottoms (° C.) | n.a. | 185 |
| T reboiler (° C.) | n.a. | 187 |
| Reflux ratio org. (g/g) | n.a. | 42.8 |
| Heat duty reboiler (kW) | n.a. | 28 |

In Comparative Example 6, the specific energy demand was 635 kW/t_{IPDA}.

In Example 6, the specific energy demand was slightly lower at 587 kW/t_{IPDA}.

In both examples (Comparative Example 6 and Example 6), the IPDA loss was the same (0.3%). In Example 6 an additional value fraction IPAA was obtained without having to significantly increase the specific energy demand of the column-setup. The IPAA recovery in Example 6 was 93.3% compared to 0% in Comparison Example 6.

1. A process for the manufacture of isophorone diamine (IPDA), comprising the steps of:
a) providing a feed stream comprising trans-IPDA, cis-IPDA, isophorone nitrile amine (IPNA), components having a lower boiling point than trans-IPDA and components having a higher boiling point than IPNA, including isophorone amino alcohol (IPAA) and components having a higher boiling point than IPAA;
b) separating the feed stream into
(i) a fraction (ii) comprising a higher mass fraction of cis-IPDA content, compared to the feed stream;
(ii) a fraction (iii) comprising a higher mass fraction of IPNA compared to the feed stream and a higher mass fraction of components having a boiling point higher than the boiling point of IPNA, including IPAA and components having a higher boiling point than IPAA, compared to the feed stream and optionally IPDA;
c) further separating fraction (iii) into
(iii) a fraction (iii-1) comprising a higher mass fraction of IPDA, compared to fraction (iii); and/or
(iv) a fraction (iii-3) comprising a higher mass fraction of IPAA, compared to fraction (iii).

2. A process according to claim 1, wherein the feed stream is separated into an additional fraction (i) comprising a higher mass fraction of trans-IPDA, compared to the feed stream.

3. A process according to at least one of claims 1 to 2, wherein the feed stream is separated into an additional fraction (iv) comprising a higher mass fraction of components having a boiling point lower than trans-IPDA compared to the feed stream.

4. A process according to claim 3, wherein fraction (iv) is further separated into an organic phase (iv-a) and an aqueous phase (iv-b).

5. A process according to at least one of claims 1 to 4, wherein fraction (iii) is further separated into one or more of the following fractions:
a fraction (iii-2) comprising a higher mass fraction of IPNA compared to fraction (iii); and/or
a fraction (iii-4) comprising a higher mass fraction of components having a boiling point higher than IPAA compared to fraction (iii).

6. A process according to at least one of claims 1 to 5, wherein fraction (iii) is separated into a fraction (iii-1); and a fraction (iii-b), which comprises fractions (iii-2), (iii-3) and (iii-4).

7. A process according to claim 6, wherein fraction (iii-b) is further separated in fractions (iii-2), (iii-3) and (iii-4) in an additional column.

8. A process according to at least one of claims 1 to 5, wherein fraction (iii) is separated into fractions (iii-1), (iii-2), (iii-3) and (iii-4) in a single column.

9. A process according to at least one of claims 1 to 5, wherein fraction (iii) is separated into a fraction (iii-a), comprising fraction (iii-2) and fraction (iii-1); and
into a fraction (iii-3); and
into a fraction (iii-4)
in a single column.

10. A process according to claims 6, 7 or 8, wherein the cis-trans ratio (CTR) of IPDA in the feed is 75:25 or less.

11. A process according to claim 9, wherein the cis-trans ratio (CTR) of IPDA in the feed is 75:25 or more.

12. A process according to claim 10 or 11, wherein the feed stream comprises:
83.4 to 91.0 percent by weight: IPDA (cis and trans);
7-10 percent by weight: water;
0.5-2.5 percent by weight: components having a boiling point lower than trans-IPDA;
0.1 to 0.4 percent by weight: IPNA;
1 to 3 percent by weight: IPAA;
0.4 to 0.7 percent by weight: components having a boiling point higher than IPAA.

13. A process according to at least of claims 1 to 10 wherein the feed stream is obtained by either (A) converting isophorone nitrile (IPN) in the presence of NH₃, H₂ and a hydrogenation catalyst in a single step or (B) converting IPN in the presence of NH₃, H₂ and a hydrogenation catalyst in at least two stages, by first converting IPN fully or partly with NH₃ in the presence of an imination catalyst to obtain isophorone nitrile imine (IPNI) and further reacting IPNI with hydrogen in the presence of a hydrogenation catalyst and optionally ammonia.

14. A process according to at least one of claims 1 to 13 wherein fraction (i) and/or fraction (ii) each comprise less than 0.2 percent by weight of IPNA.

15. A process according to claim 14 and carrying out an additional step in which the IPDA of fraction (i) is converted to isophone diisocyanate (IPDI) by conversion of IPDA of fraction (i) with phosgene to IPDI and optionally carrying out an additional step by converting IPDI with polyols to obtain polyurethanes.

16. A process according to at least one of claims 1 to 15, wherein feed stream is prepared comprising the steps of
I) converting isophorone (IP) and hydrogen cyanide (HCN) to isophorone nitrile (IPN);
II) converting IPN to isophoronenitrile imine (IPNI) in the presence of ammonia and an imination catalyst
III) converting IPNI to IPDA in the presence of hydrogen and a hydrogenation catalyst.

17. A process according to claim 16, wherein in step III) is conducted in a single fixed bed reactor.
